SEQUENCE LISTING (9 sequences)

SEQ ID NO: 1 - DNA, 1704 bp, Artificial Sequence
  Synthetic
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggatatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctctagcctcgaggccaccagcagcgcacgtctccctaagggtgggaaaatagaccaataggcagagagagtcagtgccggatcccagcgatatcaaaactagtgtgacgctgctgacccctttctttcccttctgcagggtacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcggcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctttacaaggatatccctgaattctagggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 2 - DNA, 1734 bp, Artificial Sequence
  Synthetic
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggatatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctctagcctcgaggccaccaacccctaccaactggtcggggtttgaaacagcagcgcacgtctccctaagggtgggaaaatagaccaataggcagagagagtcagtgccggatcccagcgatatcaaaactagtgtgacgctgctgacccctttctttcccttctgcagggtacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcggcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctttacaaggatatccctgaattctagggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 3 - DNA, 1799 bp, Artificial Sequence
  Synthetic
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggatatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctctagcctcgaggccaccaacccctaccaactggtcggggtttgaaactaatgcctggcttgtcgacgcatagtctgcaagtaaacccctaccaactggtcggggtttgaaacagcagcgcacgtctccctaagggtgggaaaatagaccaataggcagagagagtcagtgccggatcccagcgatatcaaaactagtgtgacgctgctgacccctttctttcccttctgcagggtacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcggcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctttacaaggatatccctgaattctagggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 4 - DNA, 1783 bp, Artificial Sequence
  Synthetic
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggatatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctctagcctcgaggccacctacttgctgatgagtccgtgaggacgaaacgagtaagctcgtccaagtaaacccctaccaactggtcggggtttgaaacagcagcgcacgtctccctaagggtgggaaaatagaccaataggcagagagagtcagtgccggatcccagcgatatcaaaactagtgtgacgctgctgacccctttctttcccttctgcagggtacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcggcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctttacaaggatatccctgaattctagggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 5 - DNA, 7356 bp, Artificial Sequence
  Synthetic
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatgagccccaagaagaagagaaaggtggaggccagcatcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactgaggcactgggtggtccataacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcagaaacaaggccgtccacctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaattccggatccggacctaagaaaaagaggaaggtggcggccgcttacccatacgatgttccagattacgctgctagctaatctagagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 6 - DNA, 7356 bp, Artificial Sequence
  Synthetic
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatgagccccaagaagaagagaaaggtggaggccagcatcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactggcgcactgggtggtcgctaacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcgcaaacaaggccgtcgccctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaattccggatccggacctaagaaaaagaggaaggtggcggccgcttacccatacgatgttccagattacgctgctagctaatctagagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 7 - DNA, 4868 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagaatggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcatactaccggtgtcgacgccaccatgcctgcagctaagcgggttaagttggatatcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactggcgcactgggtggtcgctaacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcgcaaacaaggccgtcgccctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaattccggatcccctgcagctaagcgggttaagttggatggatcctacccatacgatgttccagattacgctggatcccagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaggcaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgtaaaagcttgaattcgatctttttccctctgccaaaaattatggggacatcatgaagccccttgagcatctgacttctggctaataaaggaaatttattttcattgcaatagtgtgttggaattttttgtgtctctcactcggcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 8 - protein, 978 aa, Artificial Sequence
  Synthetic
MSPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFRNKAVHLEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNS

SEQ ID NO: 9 - protein, 978 aa, Artificial Sequence
  Synthetic
MSPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNS

(19) United States
(12) Patent Application Publication
(10) Pub. No.: US 2023/0121437 A1
(43) Pub. Date: Apr. 20, 2023

(54) RNA EDITOR-ENHANCED RNA TRANS-SPLICING

(71) Applicant: University of Massachusetts, Boston, MA (US)

(72) Inventors: Guangping Gao, Westborough, MA (US); Dan Wang, Belchertown, MA (US); Jiaming Wang, Worcester, MA (US)

(73) Assignee: University of Massachusetts, Boston, MA (US)

(21) Appl. No.: 17/768,305

(22) PCT Filed: Oct. 14, 2020
(86) PCT No.: PCT/US2020/055621

§ 371 Date: Apr. 12, 2022

Related U.S. Application Data
(60) Provisional application No. 62/982,143, filed on Feb. 27, 2020.
(60) Provisional application No. 62/915,513, filed on Oct. 15, 2019.

(51) Int. Cl. C12N 15/11; C12N 9/22; C12N 15/86; C12N 9/78; A61K 48/00; A61K 38/46; A61K 31/7105

(52) U.S. Cl. CPC C12N 15/11; C12N 9/22; C12N 15/86; C12Y 305/04004; C12N 9/78; A61K 48/0066; A61K 38/465; A61K 31/7105; C12N 2310/20; C12N 2800/80; C12N 2320/33; C12N 2750/14143; C07K 2319/09

(57) ABSTRACT

Aspects of the disclosure relate to compositions and methods for exon replacement in a cell or a subject. In some embodiments, the disclosure relates to isolated nucleic acids (and vectors, such as rAAV vectors) encoding one or more guideRNAs (gRNAs) that target an intron-exon boundary; an intronic sequence having a splice signal; and a donor sequence encoding a gene product of a gene of interest, or portion thereof. In some embodiments, compositions described herein are useful for replacing mutant exons associated with certain diseases, for example Duchen's muscular dystrophy (DMD), cystic fibrosis (CF), spinal muscular atrophy (SMA), Rett syndrome, and mucopolysaccharidosis (MPS).

### CROSS-REFERENCE TO RELATED APPLICATIONS

This application is a national stage filing under 35 U.S.C. § 371 of international PCT application PCT/US2020/055621, filed Oct. 14, 2020, which claims priority under 35 U.S.C. § 119(e) to U.S. provisional patent application, U.S. Ser. No. 62/982,143, filed Feb. 27, 2020 and U.S. Ser. No. 62/915,513, filed Oct. 15, 2019, the entire contents of each of which are incorporated herein by reference.

### REFERENCE TO A SEQUENCE LISTING SUBMITTED AS A TEXT FILE VIA EFS-WEB

This application contains a Sequence Listing which has been submitted in ASCII format via EFS-Web and is hereby incorporated by reference in its entirety. Said ASCII copy, created on Apr. 11, 2022, is named U012070131US02-SEQ-KZM and is 52,759 bytes in size.

### BACKGROUND

Precise gene editing employing the RNA-guided DNA nuclease CRISPR-Cas9 and homology-directed repair (HDR), or based on DNA base editors, has been widely used in cell cultures and animal models as research tools and for therapeutic purposes. The low HDR efficiency in post-mitotic cells and the requirement of a protospacer-adjacent motif (PAM) limit the application of Cas9-mediated DNA editing in some cases.

### SUMMARY

Aspects of the disclosure relate to compositions and methods for RNA editing. The disclosure is based, in part, on compositions, such as viral vectors (e.g., rAAV vectors), comprising CRISPR-Cas13 molecules (e.g., guide RNAs (gRNAs), nucleases, etc.) and donor molecules for spliceosome-mediated RNA trans-splicing. In some embodiments, compositions described herein are useful for treating certain diseases, for example diseases amenable to exon replacement-based therapies (e.g., diseases characterized by loss or reduction of protein function due to one or more mutations in an exon of a gene of interest).

Accordingly, in some aspects, the disclosure provides an isolated nucleic acid comprising an expression cassette encoding a pre-trans-splicing (PTS) molecule (also referred to as a PTM), wherein the PTS molecule comprises: i) one or more guide RNAs (gRNAs) that target an intron-exon boundary; ii) an intronic sequence having a splice signal; and iii) a donor sequence encoding a gene product of a gene of interest, or portion thereof.

In some embodiments, one or more gRNAs comprise one or more Cas13 direct repeats. In some embodiments, a splice signal comprises a branch point sequence (BPS).

In some embodiments, a donor sequence is an exonic sequence. In some embodiments, a donor sequence comprises an entire exon of a gene of interest. In some embodiments, the donor sequence of (iii) comprises more than one entire exon of a gene of interest.

In some embodiments, an expression cassette comprises a promoter operably linked to a nucleic acid sequence encoding a PTS molecule. In some embodiments, a promoter is an RNA polymerase II (pol II) promoter. In some embodiments, a promoter is a chicken beta-actin (CB) promoter. In some embodiments, a promoter is an inducible promoter or a tissue-specific promoter.

In some embodiments, one or more gRNAs and an intronic sequence are adjacent to one another. In some embodiments, one or more gRNAs are positioned 5′ relative to an intronic sequence. In some embodiments, one or more gRNAs are positioned 3′ relative to an intronic sequence.

In some embodiments, a donor sequence is positioned 3′ relative to an intronic sequence. In some embodiments, a donor sequence is positioned 5′ relative to an intronic sequence.

In some embodiments, an expression construct further comprises a sequence encoding a self-cleaving ribozyme. In some embodiments, a ribozyme is a hammerhead ribozyme. In some embodiments, a ribozyme-encoding sequence is positioned 5′ to one or more gRNAs.

In some embodiments, an expression construct further comprises a sequence encoding an RNA-guided nuclease. In some embodiments, an RNA-guided nuclease is a Cas13 nuclease. In some embodiments, a Cas13 nuclease is selected from the group consisting of Cas13b, Cas13d, and dCas13d nuclease. In some embodiments, a sequence encoding an RNA-guided nuclease is operably linked to a nuclear localization signal (NLS) sequence.

In some embodiments, an expression construct further comprises a sequence encoding an adenosine deaminase domain. In some embodiments, a deaminase domain is an ADAR deaminase domain (ADARDD).

In some embodiments, an expression cassette is flanked by viral vector repeat sequences. In some embodiments, viral vector repeat sequences are adeno-associated virus (AAV) inverted terminal repeats (ITRs).

In some aspects, the disclosure provides a composition comprising an isolated nucleic acid as described herein. In some embodiments, the disclosure provides a cell (e.g., a host cell) comprising an isolated nucleic acid as described herein. In some embodiments, a cell is a mammalian cell, for example a human cell.

In some embodiments, a composition further comprises an isolated nucleic acid encoding an RNA-guided nuclease. In some embodiments, an RNA-guided nuclease is a Cas13 nuclease. In some embodiments, a Cas13 nuclease is selected from the group consisting of Cas13b, Cas13d, and dCas13d nuclease. In some embodiments, a nucleic acid sequence encoding an RNA-guided nuclease is operably linked to a nuclear localization signal (NLS) sequence.

In some embodiments, a composition further comprises an isolated nucleic acid encoding an adenosine deaminase domain. In some embodiments, a deaminase domain is an ADAR deaminase domain (ADARDD).

In some aspects, the disclosure provides a composition comprising (i) a first recombinant adeno-associated virus (rAAV) particle comprising the isolated nucleic acid as described by the disclosure; and (ii) a second rAAV particle encoding an RNA-guided nuclease. In some embodiments, an RNA-guided nuclease is a Cas13 nuclease. In some embodiments, a Cas13 nuclease is selected from the group consisting of Cas13b, Cas13d, and dCas13d nuclease.

In some embodiments, a second rAAV encodes an adenosine deaminase domain. In some embodiments, the deaminase domain is an ADAR deaminase domain (ADARDD).

In some aspects, the disclosure provides a method for replacing a mutant exon of a gene of interest in a cell, the method comprising expressing in a cell having a mutant exon of a gene of interest: the isolated nucleic acid encoding a PTS molecule, wherein one or more of the gRNAs encoded by the isolated nucleic acid specifically bind to an intron-exon boundary that is 5′ relative to the mutant exon of the gene of interest; and an RNA-guided nuclease, wherein the donor sequence of the isolated nucleic acid encodes a wild-type exon of the gene of interest corresponding to the mutant exon.

In some embodiments, a mutant exon comprises one or more nucleic acid substitutions, insertions, or deletions relative to a wild-type exon.

In some embodiments, a cell is a mammalian cell. In some embodiments, a mammalian cell is a human cell. In some embodiments, a cell is in a subject. In some embodiments, a subject is a human.

In some embodiments, a gene of interest is DMD, CFTR, SMN1, SMN2, MECP2, or IDUA. In some embodiments, a gene of interest is a gene associated with cancer (e.g., an oncogene), for example a gene associated with tumor cell proliferation, metastasis, apoptosis, or resistance to therapeutic modalities (e.g., drug resistance, radiotherapy, etc.).

In some embodiments, an isolated nucleic acid and/or the RNA-guided nuclease is expressed by an rAAV.

In some embodiments, expression of an isolated nucleic acid and an RNA-guided nuclease results in translation of a full-length, wild-type gene product of a gene of interest.

In some aspects, the disclosure provides a method for treating a disease associated with a loss of protein function in a subject in need thereof, the method comprising administering to the subject: an isolated nucleic acid encoding a PTS molecule, wherein one or more of the gRNAs encoded by the isolated nucleic acid specifically bind to an intron-exon boundary that is 5′ relative to a mutant exon of a gene of interest; and an RNA-guided nuclease, wherein the subject has a disease characterized by the presence of the mutant exon in the gene of interest, and wherein the donor sequence of the isolated nucleic acid encodes a wild-type exon of the gene of interest corresponding to the mutant exon.

In some aspects, the disclosure relates to a method for treating a disease associated with a dominant negative protein function in a subject in need thereof, the method comprising administering to the subject: (i) any of the isolated nucleic acids of the disclosure, wherein one or more of the gRNAs encoded by the isolated nucleic acid specifically bind to an intron-exon boundary that is 5′ relative to a mutant exon of a gene of interest; and (ii) an RNA-guided nuclease; wherein the subject has a disease characterized by the presence of the mutant exon in the gene of interest, and wherein the donor sequence of the isolated nucleic acid encodes a wild-type exon of the gene of interest corresponding to the mutant exon.

In some aspects, the disclosure relates to a method for treating a disease associated with a gain of function protein function in a subject in need thereof, the method comprising administering to the subject: (i) any of the isolated nucleic acids of the disclosure, wherein one or more of the gRNAs encoded by the isolated nucleic acid specifically bind to an intron-exon boundary that is 5′ relative to a mutant exon of a gene of interest; and (ii) an RNA-guided nuclease; wherein the subject has a disease characterized by the presence of the mutant exon in the gene of interest, and wherein the donor sequence of the isolated nucleic acid encodes a wild-type exon of the gene of interest corresponding to the mutant exon.

In some embodiments, a mutant exon is located in one of the following genes: DMD, CFTR, SMN1, SMN2, MECP2, or IDUA. In some embodiments, a mutant exon comprises one or more nucleic acid substitutions, insertions, or deletions relative to a wild-type exon.

In some embodiments, administration of an isolated nucleic acid and an RNA-guided nuclease results in translation of a full-length, wild-type gene product of a gene of interest. In some aspects, the disclosure relates to an isolated nucleic acid comprising a pre-trans-splicing (PTS) molecule, wherein the PTS molecule comprises: i) one or more guideRNAs (gRNAs) that target an intron-exon boundary; ii) an intronic sequence having a splice signal; and iii) a donor sequence encoding a gene product of a gene of interest, or portion thereof.

### BRIEF DESCRIPTION OF DRAWINGS

FIGS. 1A-1B show the design of CRISPR-Cas13-facilitated trans-splicing. The exons with mutations located at either 3′ or 5′ could be repaired by Cas13d- or Cas13b-facilitated RNA trans-splicing. FIG. 1A shows cartoons showing the strategy of 3′ exon replacement. The gRNA locates at the 5′ of the combined PTM and Cas13d platforms are used. FIG. 1B is cartoons showing the strategy of 5′ exon replacement. The gRNA locates at the 3′ of the combined PTM and Cas13b platforms are used.

FIG. 2 shows the reporter system used to test the efficiency of RNA trans-splicing. A reporter gene containing N-terminal EGFP and C-terminal Cluc sequences, which are linked by an intron, is constructed. The mRNA sequence of C terminal EGFP are combined with gRNA of Cas13 to generate gRNA-PTM. Reporter, gRNA-PTM, and Cas13 platforms are co-transfected in HEK 293FT cells. The efficiency of RNA trans-splicing could be indicated by the percentage of EGFP+ cells.

FIG. 3 shows CRISPR-Cas13 platforms could efficiently facilitate RNA trans-splicing in vitro. Fluorescence images of traditional RNA trans-splicing strategy 24 h after transfection are shown in panel A. The PTM with a binding domain only. Fluorescence images of different Cas13 platforms-facilitated RNA trans-splicing strategy 24 h after transfection are shown in panels B1-D3. Active Cas13, dead version Cas13, and dCas13-ADARDD are used from row 1 to 3 respectively. Panels B1-B3 are images of fusion PTM with a gRNA to recruit Cas13. Panels C1-C3 are images of fusion PTM with dual-gRNAs allowing cleavage by the recruited Cas13 to remove the 5′ GpppG modification. Panels D1-D3 are images of fusion PTM with a gRNA and a hammerhead ribozyme that self-cleaves and removes the 5′ GpppG modification.

FIGS. 4A-4D show schematics depicting gene therapy for dystrophin mutations by CRISPR-Cas13-facilitated RNA Trans-splicing. FIG. 4A shows an mRNA sequence of dystrophin. Two deletion hotspots are indicated by red lines. FIG. 4B shows a 5′ exons replacement strategy by CRISPR-Cas13-facilitated RNA Trans-splicing. The gRNA with trans-splicing RNA containing wildtype exons is designed to target intron 19 or downstream introns to replace the first deletion hotspot. FIG. 4C shows a 3′ exons replacement strategy by CRISPR-Cas13-facilitated RNA Transplicing. The gRNA with trans-splicing RNA (gtsRNA) containing wildtype exons is designed to target intron 48 or downstream introns to replace part of the second deletion hotspot as the gtsRNA is within packaging size of AAV. FIG. 4D shows an internal exons replacement strategy by CRISPR-Cas13-facilitated RNA Tran-splicing to cover the whole second deletion hotspot. The targeting introns are introns outside of the deletion region.

FIGS. 5A-5B show schematics depicting gene therapy for CFTR mutations by CRISPR-Cas13-facilitated RNA Trans-splicing. FIG. 5A shows an mRNA sequence of CFTR. The most common F508del is indicated by red arrow. FIG. 5B shows a 3′ exons replacement strategy by CRISPR-Cas13-facilitated RNA Transplicing. The gRNA is designed to target intron 10 or more upstream introns.

FIGS. 6A-6B show schematics depicting gene therapy for SMN mutations by CRISPR-Cas13-facilitated RNA Trans-splicing. FIG. 6A shows a schematic representation of SMN RNA and nucleotides difference between SMN1 and SMN2. FIG. 6B shows a 3′ exons replacement strategy by CRISPR-Cas13-facilitated RNA Transplicing. The gRNA is designed to target intron 6. Trans-splicing RNA contain exon7 and exon 8 of SMN1 wildtype sequence. This design repairs SMN1 and SMN2 at the same time to restore the functional SMN protein.

FIGS. 7A-7C show schematics depicting gene therapy for MECP2 mutations by CRISPR-Cas13-facilitated RNA Trans-splicing to keep endogenous expression and regulation. FIG. 7A shows MECP2 structure showing exons and introns and alternative splicing results in two transcripts with different 3′ ends. FIGS. 7B-7C show a 3′ exons replacement strategy by CRISPR-Cas13-facilitated RNA Transplicing could replace exon 4, which covers most of MECP2 mutations in both transcripts, to keep the endogenous expression and regulation. The gRNA targets intron 3.

FIGS. 8A-8D show schematics depicting gene therapy for IDUA mutations by CRISPR-Cas13-facilitated RNA Trans-splicing. FIG. 8A shows an mRNA sequence of IDUA. The most common nonsense mutations (Q70X and W402X) are indicated by red arrows. FIG. 8B shows a 5′ exons replacement strategy by CRISPR-Cas13-facilitated RNA Transplicing. The gRNA is designed to target intron 2 or later introns to replace the exon 2 containing Q70X mutation. FIG. 8C shows a 3′ exons replacement strategy by CRISPR-Cas13-facilitated RNA Transplicing. The gRNA is designed to target intron 8 or more upstream introns to replace the exon 9 containing W402X mutation. FIG. 8D shows a 3′ exons replacement strategy by CRISPR-Cas13-facilitated RNA Transplicing with a longer trans-splicing RNA sequence to cover the two nonsense mutations at the same time. The gRNA is designed to target intron 1.

FIG. 9 shows a schematic depicting one embodiment of a plasmid encoding an rAAV vector comprising a pre-trans-splicing molecule (PTM) for replacement of a 3′ exon.

FIG. 10 shows a schematic depicting one embodiment of a plasmid encoding an rAAV vector comprising a pre-trans-splicing molecule (PTM) for replacement of a 3′ exon.

FIG. 11 shows a schematic depicting one embodiment of a plasmid encoding an rAAV vector comprising a pre-trans-splicing molecule (PTM) for replacement of a 3′ exon.

FIG. 12 shows a schematic depicting one embodiment of a plasmid encoding an rAAV vector comprising a pre-trans-splicing molecule (PTM) for replacement of a 3′ exon.

FIG. 13 shows a schematic depicting one embodiment of a plasmid encoding a viral vector comprising an RNA-guided nuclease (e.g., a Cas13 nuclease).

FIG. 14 shows a schematic depicting one embodiment of a plasmid encoding a viral vector comprising a catalytically-inactive RNA-guided nuclease (e.g., a dCas13 nuclease).

FIG. 15 shows a schematic depicting one embodiment of a plasmid encoding a viral vector comprising an RNA-guided nuclease (e.g., a Cas13 nuclease) fused to a deaminase domain (e.g., ADARDD).

### DETAILED DESCRIPTION

Aspects of the disclosure relate to compositions and methods for RNA editing. In some embodiments, the disclosure relates to an isolated nucleic acid encoding a pre-trans-splicing (PTS) molecule, wherein the PTS molecule comprises i) a guide RNA (gRNA), ii) an intronic sequence having a splice signal, and iii) a donor sequence encoding a gene of interest or portion thereof. In some embodiments, a composition further comprises an RNA-guided nuclease (e.g., an isolated nucleic acid encoding an RNA-guided nuclease, such as a Cas13 nuclease). In some embodiments, compositions described by the disclosure are useful for treating certain diseases, for example diseases amenable to exon replacement therapy (e.g., Duchene's muscular dystrophy, cystic fibrosis, spinal muscular atrophy (SMA), Rett syndrome, mucopolysaccharidosis (MPS), etc.).

CRISPR-Cas-Facilitated Trans-Splicing

Aspects of the disclosure relate to compositions and methods for RNA-guided gene editing, also referred to as “programmable RNA editing.” Programmable RNA editing generally does not permanently alter the genetic information of a cell, and therefore has a potentially less concerning safety profile as a gene therapy approach. Programmable RNA editing typically involves delivery of at least two components to a cell: an RNA-guided nuclease and a guide-RNA to direct the activity of the nuclease, to a cell or subject.

In some embodiments, a nucleic acid encodes an RNA-guided nuclease (RGN). As used herein, the terms “endonuclease” and “nuclease” refer to an enzyme that cleaves a phosphodiester bond or bonds within a polynucleotide chain. Nucleases may be naturally occurring or genetically engineered. Genetically engineered nucleases are particularly useful for genome editing and are generally classified into four families: zinc finger nucleases (ZFNs), transcription activator-like effector nucleases (TALENs), meganucleases (e.g., engineered meganucleases) and CRISPR-associated proteins (Cas nucleases).

In some embodiments, an RNA-guided nuclease is a CRISPR-associated protein (e.g., a Cas nuclease). The term “CRISPR” refers to “clustered regularly interspaced short palindromic repeats,” which are DNA loci containing short repetitions of base sequences. CRISPR loci form a portion of a prokaryotic adaptive immune system that confers resistance to foreign genetic material. Each CRISPR loci is flanked by short segments of “spacer DNA”, which are derived from viral genomic material. In the Type II CRISPR system, spacer DNA hybridizes to transactivating RNA (tracrRNA) and is processed into CRISPR-RNA (crRNA) and subsequently associates with CRISPR-associated nucleases (Cas nucleases) to form complexes that recognize and degrade foreign DNA. In the Type VI CRISPR system, CRISPR-RNA (crRNA) associates with CRISPR-associated nucleases (Cas nucleases) to form complexes that recognize and degrade RNA. Examples of CRISPR nucleases include, but are not limited to Cas9, dCas9, Cas6, Cas13, CasRX, Cpf1, and variants thereof.

In some embodiments, a Cas protein is modified (e.g., genetically engineered) to lack nuclease activity. For example, “dead” Cas13 (dCas13) protein binds to a target locus but does not cleave said locus. In some embodiments, a Cas protein or variant thereof does not exceed the packaging capacity of a viral vector, such as a lentiviral vector or an adeno-associated virus (AAV) vector, for example as described by Ran et al., (2015) Nature. 520(7546); 186-91. For example, in some embodiments, a nucleic acid encoding a Cas protein is less than about 4.6 kb in length.

In some embodiments, an RNA-guided nuclease is a Type VI CRISPR nuclease (e.g., a nuclease that binds to and/or cleaves RNA, such as pre-mRNA or mRNA). In some embodiments, an RNA-guided nuclease is a Cas13 nuclease. In some embodiments, the Cas13 nuclease is a Leptotrichia buccalis, Leptotrichia shahii, Ruminococcus flavefaciens, Bergeyella zoohelcum, Prevotella buccae, or Listeria seeligeri Cas13 nuclease. In some embodiments, the independence of endogenous nucleic acid repair mechanisms and target sequence context of CRISPR-Cas13 markedly expand the feasibility of targets manipulation, compared with CRISPR-Cas9.

The disclosure is based, in part, on Cas13 nucleases (and variants thereof) which are configured to mediate trans-splicing of a donor sequence (e.g., a donor exon) in order to replace a mutant exon (e.g., an exon of a gene of interest having one or more mutations, substitutions, insertions, deletions, etc. relative to the corresponding wild-type exon of the gene of interest). In some embodiments, spliceosome-mediated RNA trans-splicing, modifies mRNA by replacing a portion of the target endogenous pre-mRNA sequence (e.g., a mutant exon of a gene of interest) with exogenous RNA (e.g., as delivered by a pre-trans-splicing molecule, PTM) in a trans-splicing event. In some embodiments, trans-splicing editing repairs a broader range of genetic mutations, including not only point mutations, but also indels or other complicated mutations, relative to other gene therapy technologies. The repair mRNA product is subjected to the endogenous transcriptional regulation of the cell, which is an advantage. The reduction of delivered gene size also makes it more feasible for viral vector delivery.

In some embodiments, a Cas13 nuclease is configured to replace a 5′ exon. In some embodiments, a Cas13 nuclease is configured to replace a 3′ exon. In some embodiments, a Cas13 nuclease is a Cas13b nuclease. In some embodiments, a Cas13 nuclease is a Cas13d nuclease. In some embodiments, a Cas13 nuclease is a dead Cas13 nuclease (e.g., a Cas13 nuclease that lacks the ability to cleave nucleic acids). In some embodiments, a Cas13 nuclease comprises the amino acid sequence set forth in SEQ ID NO: 8. In some embodiments, a dCas13 nuclease comprises the amino acid sequence set forth in SEQ ID NO: 9.

A variant of an RGN (e.g., a variant of a Cas13 nuclease) may comprise or consist of a nucleic acid sequence that comprises one or more substitutions, insertions, and/or deletions relative to a wild-type RGN nucleic acid sequence (e.g., a wild-type Cas13 protein, such a set forth in SEQ ID NO: 8). In some embodiments, a variant of an RGN comprises or consists of a nucleic acid sequence that is at least 50% (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, 99.95%, 99.99%, or more) identical to a wild-type RGN nucleic acid sequence. In some embodiments, a variant of an RGN comprises or consists of an amino acid sequence that is at least 50% (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, 99.95%, 99.99%, or more) identical to a wild-type RGN amino acid sequence. In some embodiments, an RGN is a “dead” RGN, such as an RGN that retains binding functionality but lacks nuclease activity (e.g., is catalytically dead).

The terms “percent identity,” “sequence identity,” “% identity,” “% sequence identity,” and % identical,” as they may be interchangeably used herein, refer to a quantitative measurement of the similarity between two sequences (e.g., nucleic acid or amino acid). The percent identity of genomic DNA sequence, intron and exon sequence, and amino acid sequence between humans and other species varies by species type, with chimpanzee having the highest percent identity with humans of all species in each category.

Calculation of the percent identity of two nucleic acid sequences, for example, can be performed by aligning the two sequences for optimal comparison purposes (e.g., gaps can be introduced in one or both of a first and second nucleic acid sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences.

The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Atschul, S. F. et al., J. Molec. Biol., 215, 403 (1990)).

When a percent identity is stated, or a range thereof (e.g., at least, more than, etc.), unless otherwise specified, the endpoints shall be inclusive and the range (e.g., at least 70% identity) shall include all ranges within the cited range (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) and all increments thereof (e.g., tenths of a percent (e.g., 0.1%), hundredths of a percent (e.g., 0.01%), etc.).

In some embodiments, an RNA-guided nuclease (e.g., a Cas13d nuclease, dCas13d nuclease, Cas13b nuclease, etc.) is fused to a base editing domain. Generally, a “base editing domain” refers to an enzyme (or portion thereof) capable of converting (e.g., substituting) one nucleotide of a nucleic acid to a different nucleotide, for example creating point-mutations in a nucleic acid. A base editing domain may be an adenine base editing enzyme, cytidine base editing enzyme, uracil base editing enzyme, etc. In some embodiments, a base editing domain comprises an adenine base editing enzyme. Examples of adenosine deaminase base editing domains include ADAR1, ADAR2, etc., for example as described by Wang et al., (2018) Biochemistry 57(10):1640-1651. In some embodiments, a base editing domain is an adenosine deaminase domain (ADARDD). In some embodiments, an adenosine deaminase domain (e.g., ADARDD) is fused to a Cas13d nuclease or a Cas13b nuclease, for example as described by Cox et al., (2017) Science, 358(6366):1019-1027.

Aspects of the disclosure relate to isolated nucleic acids comprising an expression construct encoding pre-trans-splicing molecules (PTMs) having one or more (e.g., 1, 2, 3, 4, 5, or more) guide RNAs (gRNAs). For the purpose of genome editing, the CRISPR system can be modified to combine the tracrRNA and crRNA into a single guide RNA (sgRNA) or just (gRNA). As used herein, the terms “guide RNA”, “gRNA”, and “sgRNA” refer to a polynucleotide sequence that is complementary to a target sequence in a cell and associates with a Cas nuclease, thereby directing the Cas nuclease to the target sequence. In some embodiments, a gRNA (e.g., sgRNA) ranges between 1 and 30 nucleotides in length. In some embodiments, a gRNA (e.g., sgRNA) ranges between 5 and 25 nucleotides in length. In some embodiments, a gRNA (e.g., sgRNA) ranges between 10 and 22 nucleotides in length. In some embodiments, a gRNA (e.g., sgRNA) ranges between 14 and 24 nucleotides in length. In some embodiments, a Cas protein and a guide RNA (e.g., sgRNA) are expressed from the same vector. In some embodiments, a Cas protein and a guide RNA (e.g., sgRNA) are expressed from separate vectors (e.g., two or more vectors).

In some embodiments, the disclosure relates to PTMs comprising i) one or more guideRNAs (gRNAs) that target an intron-exon boundary (e.g., target a RNA transcript that comprises an intron-exon boundary, for example a RNA transcript of a gene of interest); ii) an intronic sequence having a splice signal; and iii) a donor sequence encoding a gene product of a gene of interest, or portion thereof.

A gRNA may be any sequence comprising sufficient complementarity to hybridize with a sequence of a target gene. For example, a gRNA generally comprises a region of complementarity that allows it to hybridize (e.g., specifically bind) to a sequence of a gene of interest or target gene. As used herein, a “gene of interest” or “target gene” refers to the gene which is targeted for manipulation (e.g., removal, correction, replacement, editing, etc.). The terms “complementary” and “complementarity,” as may be used interchangeably herein, refer a property of a nucleotide (e.g., A, C, G, T, U) in a nucleic acid (e.g., RNA, DNA) in a strand (e.g., oligonucleotide) to pair with another particular nucleotide in a nucleic acid strand of the opposite orientation (e.g., strands running parallel, but in the reverse direction (i.e., 5′-3′ aligns with 3′-5′, and 3′-5′ with 5′-3′)) (i.e., Watson-Crick base-pairing rules). With respect to deoxyribonucleic acids (DNA) the base pairings which are complementary are adenine (A) and thymine (T) (e.g., A with T, T with A) and guanine (G) and Cytosine (C) (e.g., G with C, C with G) and with respect to ribonucleic acid (RNA) the base pairings which are complementary are A and uracil (U) (e.g., A with U, U with A) and G and C (e.g., G with C, C with G). This occurs because of the ability of each base pair to form an equivalent number of hydrogen bonds with its complementary base (e.g., A-T/U, T/U-A, C-G, G-C), for example the bond between guanine and cytosine shares three hydrogen bonds compared to the A-T/U bond which always shares two hydrogen bonds.

When every base in at least one strand of a pair of nucleic acids is found opposite its complementary base pair, such strand is considered fully complementary to its sequence in the other strand. When one, or more, bases of such a strand is found in a position where it is opposite any other base excepting its complementary base pair, that base is considered “mis-matched” and the strand is considered partially complementary. Accordingly, strands can be varying degrees of partially complementary, until no bases align, at which point they are non-complementary. In some embodiments, a gRNA is at least 50% (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, 99.95%, 99.99%, or more) complementary, with a sequence of a target gene.

Additionally, as disclosed hereinabove, a gRNA may be less than 100% complementary, accordingly in some embodiments, a gRNA comprises mismatches (e.g., non-complementary bases) to a sequence in a target gene. In some embodiments, a gRNA comprises at least 1 (e.g., 1, 2, 3, 4, 5, 6, 7, 8, 9, 10, 11, 12, 13, 14, 15, 16, 17, 18, 19, 20, 21, 22, 23, 24, 25, or more) mismatches (e.g., non-complementary bases).

In some embodiments, the gRNA targets an intron-exon boundary in a target gene. Intron-exon boundaries will vary depending on the target gene and exon targeted, however, ascertaining the sequence of such boundary will readily be understood by the skilled artisan without undue experimentation.

For example, in some embodiments, a target gene is DMD, CFTR, SMN1, SMN2, MECP2, IDUA, DYSF, MYO7A, ABC1, ABCA4, FVIII, FANCA, PDHA1, GAA, GBA1, PAH, and/or BCKDHA. In some embodiments, a target gene is DMD. In some embodiments, a target gene is CFTR. In some embodiments, a target gene is SMN1. In some embodiments, a target gene is SMN2. In some embodiments, a target gene is MECP2. In some embodiments, a target gene is IDUA. In some embodiments, a target gene is DYSF. In some embodiments, a target gene is MYO7A. In some embodiments, a target gene is ABC1. In some embodiments, a target gene is ABCA4. In some embodiments, a target gene is FVIII. In some embodiments, a target gene is FANCA. In some embodiments, a target gene is PDHA1. In some embodiments, a target gene is GAA. In some embodiments, a target gene is GBA1. In some embodiments, a target gene is PAH. In some embodiments, a target gene is BCKDHA.

In some embodiments, a target gene is DMD (NCBI Gene ID: 1756, updated on 10 Oct. 2020; RefSeqGene: NG_012232.1). In some embodiments, a target gene is CFTR (NCBI Gene ID: 1080, updated on 11 Oct. 2020; RefSeqGene: NG_016465.4 RefSeqGene). In some embodiments, a target gene is SMN1 (NCBI Gene ID: 6606, updated on 11 Oct. 2020; RefSeqGene: NG_008691.1). In some embodiments, a target gene is SMN2 (NCBI Gene ID: 6607, updated on 4 Oct. 2020; RefSeqGene: NG_008728.1). In some embodiments, a target gene is MECP2 (NCBI Gene ID: 4204, updated on 4 Oct. 2020; RefSeqGene: NG_007107.2). In some embodiments, a target gene is IDUA (NCBI Gene ID: 3425, updated on 22 Aug. 2020; RefSeqGene: NG_008103.1). In some embodiments, a target gene is DYSF (NCBI Gene ID: 8291, updated on 22 Aug. 2020; RefSeqGene: NG_008694.1). In some embodiments, a target gene is MYO7A (NCBI Gene ID: 4647, updated on 20 Sep. 2020; RefSeqGene: NG_009086.2). In some embodiments, a target gene is ABC1 (NCBI Gene ID: 19, updated on 11 Oct. 2020; RefSeqGene: NG_007981.1). In some embodiments, a target gene is ABCA4 (NCBI Gene ID: 24, updated on 4 Oct. 2020; RefSeqGene: NG_009073.1). In some embodiments, a target gene is FVIII (NCBI Gene ID: 2157, updated on 4 Oct. 2020; RefSeqGene: NG_011403.1). In some embodiments, a target gene is FANCA (NCBI Gene ID: 2175, updated on 22 Aug. 2020; RefSeqGene: NG_011706.1). In some embodiments, a target gene is PDHA1 (NCBI Gene ID: 5160, updated on 12 Sep. 2020; RefSeqGene: NG_016781.1). In some embodiments, a target gene is GAA (NCBI Gene ID: 2548, updated on 4 Oct. 2020; RefSeqGene: NG_009822.1). In some embodiments, a target gene is GBA1 (NCBI Gene ID: 2629, updated on 4 Oct. 2020; RefSeqGene: NG_009783.1). In some embodiments, a target gene is PAH (NCBI Gene ID: 5053, updated on 4 Oct. 2020; RefSeqGene: NG_008690.2). In some embodiments, a target gene is BCKDHA (NCBI Gene ID: 593, updated on 4 Oct. 2020; RefSeqGene: NG_013004.1).

In some embodiments, a gRNA targets an intron-exon boundary of any of the target genes disclosed herein. In some embodiments, a gRNA targets an intron-exon boundary of any of the transcripts of the genes disclosed herein.

In some embodiments, a donor sequence comprises any of the target genes, fragments thereof, or sequences which contain modifications of the target genes as disclosed herein.

Typically, a guide RNA (e.g., a gRNA or sgRNA) hybridizes (e.g., binds specifically to, for example by Watson-Crick base pairing) to a target sequence and thus directs the CRISPR/Cas protein to the target sequence. In some embodiments, a guide RNA hybridizes to (e.g., targets) a nucleic acid sequence encoding an intron-exon boundary (e.g., intron-exon splice junctions). Examples of nucleic acid sequences encoding intron-exon boundaries include but are not limited to AG/GTA, /GTAAGT, RG/GTGAG and AG/GTXXGT, where R=A or G and X=A, T, G, or C. An intron-exon boundary may vary in length. In some embodiments, a boundary is between about 10 and about 200 nucleotides in length. In some embodiments, a boundary is between about 100 and 500 nucleotides in length. In some embodiments, a boundary is more than 500 nucleotides in length (e.g., 600, 800, 1000, 5000, etc. nucleotides in length). The skilled artisan recognizes that intron-exon boundaries may vary between two given genes; however, methods of identifying or predicting intron-exon boundary sequences are known in the art, for example as described by Albertson and Thliveris (2001) Curr Protoc Hum Genet. Chapter 6: Unit 6.4, and Ceccherini et al., (1996) Methods. 9(1):98-105.

In some embodiments, a gRNA targets an intron-exon boundary of a gene of interest. As used herein, a “gene of interest” or “target gene” refers to the gene which is targeted for manipulation (e.g., removal, correction, replacement, editing, etc.). In some embodiments, the gene of interest comprises an exon having one or more mutations (e.g., substitutions, insertions, deletions, etc.) relative to a wild-type allele of the gene of interest. In some embodiments, such an exon is referred to as a “mutant exon.” In some embodiments, a gene of interest having one or more mutant exons is associated with causing (or predisposing) a subject to have a disease or disorder characterized by a reduction or loss of protein function. Examples of diseases characterized by mutant exons associated with causing disease include but are not limited to Duchene's muscular dystrophy (DMD), cystic fibrosis (CF), spinal muscular atrophy (SMA), Rett syndrome, and mucopolysaccharidosis (MPS). An intron-exon boundary (e.g., an exon-intron boundary targeted by a composition described herein, for example a PTM) may be 5′ (“upstream”) or 3′ (“downstream”) relative to a mutant exon (e.g., an exon targeted for replacement by a composition described here, for example a PTM).

In some embodiments, a disease or disorder results from a mutation in a target gene. In some embodiments a target gene has one or more (e.g., 1, 2, 3, 4, 5, 6, 7, 8, 9, 10, 15, 20, 50, 100, or more) mutations. In some embodiments each of the one or more mutations is a missense mutation, nonsense mutation, deletion, or insertion. In some embodiments, one or more mutations in a target gene results in the production of a truncated gene product (e.g., truncated protein) after translation of the target gene. In some embodiments, a truncated gene product is at least 1, 2, 3, 4, 5, 10, 15, 20, 50, 100, 200, 500, 1000, 5000, or more amino acids shorter than a gene product translated from a wildtype or full length target gene (e.g., a target gene not having one or more mutations). Examples of target genes include but are not limited to DMD, CFTR, SMN1, SMN2, MECP2, and/or IDUA. In some embodiments, a target gene comprises DMD. In some embodiments, a target gene comprises CFTR. In some embodiments, a target gene comprises SMN1. In some embodiments, a target gene comprises SMN2. In some embodiments, a target gene comprises MECP2. In some embodiments, a target gene comprises IDUA. In some embodiments, a target gene comprises DYSF. In some embodiments, a target gene comprises MYO7A. In some embodiments, a target gene comprises ABC1. In some embodiments, a target gene comprises ABCA4. In some embodiments, a target gene comprises FVIII. In some embodiments, a target gene comprises FANCA. In some embodiments, a target gene comprises PDHA1. In some embodiments, a target gene comprises GAA. In some embodiments, a target gene comprises GBA1. In some embodiments, a target gene comprises PAH. In some embodiments, a target gene comprises BCKDHA.

In some embodiments, a target gene comprises DMD (NCBI Gene ID: 1756, updated on 10 Oct. 2020; RefSeqGene: NG_012232.1). In some embodiments, a target gene comprises CFTR (NCBI Gene ID: 1080, updated on 11 Oct. 2020; RefSeqGene: NG_016465.4 RefSeqGene). In some embodiments, a target gene comprises SMN1 (NCBI Gene ID: 6606, updated on 11 Oct. 2020; RefSeqGene: NG_008691.1). In some embodiments, a target gene comprises SMN2 (NCBI Gene ID: 6607, updated on 4 Oct. 2020; RefSeqGene: NG_008728.1). In some embodiments, a target gene comprises MECP2 (NCBI Gene ID: 4204, updated on 4 Oct. 2020; RefSeqGene: NG_007107.2). In some embodiments, a target gene comprises IDUA (NCBI Gene ID: 3425, updated on 22 Aug. 2020; RefSeqGene: NG_008103.1). In some embodiments, a target gene comprises DYSF (NCBI Gene ID: 8291, updated on 22 Aug. 2020; RefSeqGene: NG_008694.1). In some embodiments, a target gene comprises MYO7A (NCBI Gene ID: 4647, updated on 20 Sep. 2020; RefSeqGene: NG_009086.2). In some embodiments, a target gene comprises ABC1 (NCBI Gene ID: 19, updated on 11 Oct. 2020; RefSeqGene: NG_007981.1). In some embodiments, a target gene comprises ABCA4 (NCBI Gene ID: 24, updated on 4 Oct. 2020; RefSeqGene: NG_009073.1). In some embodiments, a target gene comprises FVIII (NCBI Gene ID: 2157, updated on 4 Oct. 2020; RefSeqGene: NG_011403.1). In some embodiments, a target gene comprises FANCA (NCBI Gene ID: 2175, updated on 22 Aug. 2020; RefSeqGene: NG_011706.1). In some embodiments, a target gene comprises PDHA1 (NCBI Gene ID: 5160, updated on 12 Sep. 2020; RefSeqGene: NG_016781.1). In some embodiments, a target gene comprises GAA (NCBI Gene ID: 2548, updated on 4 Oct. 2020; RefSeqGene: NG_009822.1). In some embodiments, a target gene comprises GBA1 (NCBI Gene ID: 2629, updated on 4 Oct. 2020; RefSeqGene: NG_009783.1). In some embodiments, a target gene comprises PAH (NCBI Gene ID: 5053, updated on 4 Oct. 2020; RefSeqGene: NG_008690.2). In some embodiments, a target gene comprises BCKDHA (NCBI Gene ID: 593, updated on 4 Oct. 2020; RefSeqGene: NG_013004.1).

In some embodiments, the one or more mutations in the target gene occur in the DNA encoding the gene, or in a transcript (e.g., an mRNA transcript) of the gene. In some embodiments, the one or more mutations occur in an intron (e.g., one or more introns) of the target gene. In some embodiments, the one or more mutations occur in an exon (e.g., one or more exons) of a target gene. In some embodiments, a target gene comprises one or more mutations in both introns and an exons. The term “mutation,” as may be used herein, refers to a change, alteration, or modification to a nucleotide in a nucleic acid as compared to its wild-type sequence. For example, without limitation, mutations may include substitutions, insertions, deletions, or any combination of the same. In some embodiments, there at least one mutation. In some embodiments, there are more than one mutation. In some embodiments, where there is more than one mutation, the mutations are distinct (e.g., not of the same type (e.g., substitutions, insertions, deletions)). In some embodiments, where there is more than one mutation, the mutations are the same (e.g., not of the same type (e.g., substitutions, insertions, deletions)). Additionally, in some embodiments, the mutations result in a frameshift.

For example, in some embodiments, the DMD gene may have any one or more of exons 45-55 deleted. In some embodiments, the CFTR gene may have a mutation resulting in a mutation at position 508 of the wild-type amino acid sequence. In some embodiments, the mutation results in a deletion of the amino acid at position 508 in the wild-type sequence. In some embodiments, the mutation is known as delta F508. In some embodiments, the SMN1 and/or SMN2 gene has a deletion of exon 7. In some embodiments, the MECP2 gene results in a mutation in the amino acid sequence relative to the wild-type protein. In some embodiments, the mutation results in at least one of the following mutations: R168X; R255X; R270X; T158M and/or R306C relative to the wild-type amino acid sequence of the amino acid sequence encoded by MECP2. In some embodiments, the IDUA gene results in a a mutation in the amino acid sequence relative to the wild-type protein. In some embodiments, the mutation results in at least one of the following mutations: Q70X; W402X; D315Y; P533R; R621X; R628X; S633L; R162I; and/or G208D relative to the wild-type amino acid sequence of the amino acid sequence encoded by IDUA or mutations of 1352delG and/or 1952del25 bp of the nucleotide sequence. In some embodiments, a mutation results in a mutation of p.Gln832X and/or c.663+1G>C of the wild-type amino acid sequence encoded by DYSF. In some embodiments, at least one of the following mutations is present in a MYO7A gene: 0.77C>A*;c.395C>T; c.721C>G; c.1097T>C; c.3134T>C/; c.5507T>C; c.3652G>A; c.3719G>A; c.4475C>T; c.6610G>C; c.1884C>A; c.5581C>T; c.655_660del; c.986dupG; c.3764delA; c.4297delC; c.5835_5838delCTTT; c.6025delG*; and/or c.2283-1G>T. In some embodiments, a mutation in ABC/results in a R527W mutation in the amino acid sequence as compared to the wild-type. In some embodiments, a mutation occurs in exon 1 of FVIII. In some embodiments, a mutation occurs in exon 10 and/or 11 of PDHA1. In some embodiments, a mutation occurs in exon 18 of GAA. In some embodiments, a mutation occurs in exon 11 of BCKDHA.

In some embodiments, a pre-trans-splicing molecule (PTM) comprises one or more intronic sequences. Generally, an “intronic sequence” refers to a nucleic acid sequence that is removed from an RNA transcript, such as a pre-mRNA transcript, during RNA splicing (e.g., the intronic sequence is not a part of the final mRNA transcript that is translated into a protein). An intronic sequence generally comprises a splice donor site, a branch point sequence (BPS), which is a particular nucleotide sequence near the 3′ end of the intron that becomes covalently linked to the 5′ end of the intron during the splicing process, generating a branched (lariat) intron, and a splice acceptor site. The splice donor site usually includes an almost invariant sequence GU at the 5′ end of the intron, and the splice acceptor site usually contains an almost invariant AG sequence at the 3′ end of the intron. Nuclear pre-mRNA intron sequences are typically highly variable. Identification of exon-intron splice junctions is described, for example by Gao et al., (2008) Nucleic Acids Res. 36(7): 2257-2267.

In some embodiments, a pre-trans-splicing molecule (PTM) comprises a donor sequence. As used herein, a “donor sequence” refers to a nucleic acid sequence encoding a gene product (e.g., a peptide, protein or fragment thereof, inhibitory nucleic acid, etc.) that, upon contact of the PTM molecule with the target sequence of the gene of interest, is spliced together with the pre-mRNA of the gene of interest, replacing a mutant exon of the gene of interest. In some embodiments, a donor sequence comprises a nucleic acid sequence encoding an exon corresponding to a wild-type version of the mutant exon of the gene of interest. In some embodiments, a donor sequence lacks (e.g., does not comprise) any mutations (e.g., substitutions, insertions, deletions, etc.) relative to a wild-type nucleic acid sequence from which the donor sequence is derived.

In some embodiments, a pre-trans-splicing molecule (PTM) comprises a nucleic acid sequence encoding one or more steric blocking groups. A “steric blocking group” refers to a gene product (e.g., peptide, RNA molecule, etc.) that prevents or inhibits cleavage of one or more gRNAs by a spliceosome or RNA-guided nuclease. Without wishing to be bound by any particular theory, inclusion of a steric blocking group allows for preservation (e.g., no cleavage) of a 5′ gRNA sequence, thereby improving efficiency of PTM-mediated trans-splicing and/or exon replacement. In some embodiments, a steric blocking group is a ribozyme. In some embodiments, the ribozyme is a self-cleaving ribozyme, for example a hammerhead ribozyme. In some embodiments, a ribozyme is an RNasP. In some embodiments, a ribozyme is a peptidyl transferase 23SrRNA. In some embodiments, a ribozyme is a GIR1 branching ribozyme. In some embodiments, a ribozyme is a leadzyme. In some embodiments, a ribozyme is Group I intron. In some embodiments, a ribozyme is a Group II intron. In some embodiments, a ribozyme is hairpin ribozyme. In some embodiments, a ribozyme is HDV ribozyme. In some embodiments, a ribozyme is a VS ribozyme. In some embodiments, a ribozyme is a mammalian CPEB3 ribozyme. In some embodiments, a ribozyme is CoTC ribozyme. In some embodiments, a ribozyme is glmS ribozyme. Hammerhead ribozymes are described, for example by Scott et al., (2013) Prog Mol Biol Transl Sci. 120: 1-23.

The positioning of components of a PTM may vary, for example depending upon whether a 5′ exon or a 3′ exon is to be replaced. In some embodiments, one or more gRNAs and an intronic sequence are adjacent to one another. In some embodiments, one or more gRNAs are positioned 5′ relative to an intronic sequence. In some embodiments, one or more gRNAs are positioned 3′ relative to an intronic sequence. In some embodiments, a donor sequence is positioned 3′ relative to an intronic sequence. In some embodiments, a donor sequence is positioned 5′ relative to an intronic sequence. In some embodiments, a steric blocking group (e.g., a ribozyme-encoding sequence) is positioned 5′ to one or more gRNAs.

Isolated Nucleic Acids

Aspects of the disclosure relate to nucleic acids (e.g., isolated nucleic acids) encoding a pre-trans-splicing (PTS) molecule and/or an RNA-guided nuclease.

A “nucleic acid” sequence refers to a DNA or RNA sequence. In some embodiments, proteins and nucleic acids of the disclosure are isolated. As used herein, the term “isolated” means artificially produced. As used herein, with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable (e.g., amenable to, or capable of manipulation by the skilled artisan) by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.). In some embodiments, a nucleic acid is an in vitro transcribed (IVT) mRNA molecule. In some embodiments, a nucleic acid is a closed-ended linear duplex (CELiD) DNA.

As used herein, a nucleic acid sequence (e.g., coding sequence) and regulatory sequences are said to be “operably linked” when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly, two or more coding regions are operably linked when they are linked in such a way that their transcription from a common promoter results in the expression of two or more proteins having been translated in frame.

A “promoter” refers to a DNA sequence recognized by the synthetic machinery of the cell, or introduced synthetic machinery, required to initiate the specific transcription of a gene. Consistent with the definition hereinabove, the phrases “operatively linked,” “operatively positioned,” “under control,” or “under transcriptional control” means that the promoter is in the correct location and orientation in relation to the nucleic acid to control RNA polymerase initiation and expression of the gene. In some embodiments, an expression cassette or transgene comprises a nucleic acid sequence encoding an RGN (e.g., a Cas13d nuclease) operably linked to a promoter. In some embodiments, an expression cassette or transgene comprises a nucleic acid sequence encoding a pre-trans-splicing molecule (PTM) as described herein operably linked to a promoter. Generally, a promoter can be a constitutive promoter, inducible promoter, or a tissue-specific promoter. In some embodiments, a promoter is a constitutive promoter, in some embodiments, a promoter is an inducible promoter. In some embodiments, a promoter is a tissue-specific promoter.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the (3-actin promoter, the phosphoglycerol kinase (PGK) promoter, and the EF1α promoter [Invitrogen]. In some embodiments, a promoter is an RNA pol III promoter. In some embodiments, a promoter is an RNA pol III promoter, such as U6 or H1. In some embodiments, a promoter is an RNA pol II promoter. In some embodiments, a nucleic acid encoding an RGN is operably linked to a CB6 promoter. In some embodiments, a nucleic acid sequence encoding a PTM expression cassette is operably linked to an RNA pol II promoter. In some embodiments, a constitutive promoter is any of the constitutive promoters mentioned herein.

Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only. In some embodiments, an inducible promoter is any of the inducible promoters mentioned herein.

In another embodiment, the native promoter for the transgene will be used. The native promoter may be preferred when it is desired that expression of the transgene should mimic the native expression. The terms “native” and “wild-type,” as may be used interchangeably herein, are terms of art understood by skilled artisans and mean the typical form of an item, organism, strain, gene, or characteristic as it occurs in nature as distinguished from isolated, engineered, mutant, or variant forms.

The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: retinoschisin proximal promoter, interphotoreceptor retinoid-binding protein enhancer (RS/IRBPa), rhodopsin kinase (RK), liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (α-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan. In some embodiments, a regulatory sequence is any of the regulatory sequences disclosed herein.

In some embodiments, a nucleic acid sequence encoding an RNA-guided nuclease comprises a nuclear localization signal (NLS). An NLS is an amino acid sequence that ‘tags’ a protein for import into the cell nucleus by nuclear transport. Examples of NLS are described, for example by Lange et al., (2007) J Biol Chem. 282(8): 5101-5105.

In some aspects, the disclosure relates to isolated nucleic acids comprising a transgene encoding a PTM expression cassette or an RGN expression cassette, and one or more miRNA binding sites. Without wishing to be bound by any particular theory, incorporation of miRNA binding sites into gene expression constructs allows for regulation of transgene expression (e.g., inhibition of transgene expression) in cells and tissues where the corresponding miRNA is expressed. In some embodiments, incorporation of one or more miRNA binding sites into a transgene allows for de-targeting of transgene expression in a cell-type specific manner. In some embodiments, one or more miRNA binding sites are positioned in a 3′ untranslated region (3′ UTR) of a transgene, for example between the last codon of a nucleic acid sequence encoding an RGN (e.g., a Cas13 protein) and a poly A sequence, or the last nucleotide and a poly A sequence of a PTM expression cassette.

In some embodiments, a transgene comprises one or more (e.g., 1, 2, 3, 4, 5, or more) miRNA binding sites that de-target expression of the RGN or the PTM expression cassette from central nervous system (CNS) cells, such as neurons.

In some embodiments, a transgene comprises one or more (e.g., 1, 2, 3, 4, 5, or more) miRNA binding sites that de-target expression of the RGN or the PTM expression cassette from immune cells (e.g., antigen presenting cells (APCs), such as macrophages, dendrites, etc.). Incorporation of miRNA binding sites for immune-associated miRNAs may de-target transgene expression from antigen presenting cells and thus reduce or eliminate immune responses (cellular and/or humoral) produced in the subject against products of the transgene, for example as described in US 2018/0066279, the entire contents of which are incorporated herein by reference.

As used herein an “immune-associated miRNA” is an miRNA preferentially expressed in a cell of the immune system, such as an antigen presenting cell (APC). In some embodiments, an immune-associated miRNA is an miRNA expressed in immune cells that exhibits at least a 2-fold, 3-fold, 4-fold, 5-fold, 6-fold, 7-fold, 8-fold, 9-fold, 10-fold higher level of expression in an immune cell compared with a non-immune cell (e.g., a control cell, such as a HeLa cell, HEK293 cell, mesenchymal cell, etc.). In some embodiments, the cell of the immune system (immune cell) in which the immune-associated miRNA is expressed is a B cell, T cell, Killer T cell, Helper T cell, γδ T cell, dendritic cell, macrophage, monocyte, vascular endothelial cell. or other immune cell. In some embodiments, the cell of the immune system is a B cell expressing one or more of the following markers: B220, BLAST-2 (EBVCS), Bu-1, CD19, CD20 (L26), CD22, CD24, CD27, CD57, CD72, CD79a, CD79b, CD86, chB6, D8/17, FMC7, L26, M17, MUM-1, Pax-5 (BSAP), and PC47H. In some embodiments, the cell of the immune system is a T cell expressing one or more of the following markers: ART2, CD1a, CD1d, CD11b (Mac-1), CD134 (OX40), CD150, CD2, CD25 (interleukin 2 receptor alpha), CD3, CD38, CD4, CD45RO, CD5, CD7, CD72, CD8, CRTAM, FOXP3, FT2, GPCA, HLA-DR, HML-1, HT23A, Leu-22, Ly-2, Ly-m22, MICG, MRC OX 8, MRC OX-22, OX40, PD-1 (Programmed death-1), RT6, TCR (T cell receptor), Thy-1 (CD90), and TSA-2 (Thymic shared Ag-2). In some embodiments, the immune-associated miRNA is selected from: miR-15a, miR-16-1, miR-17, miR-18a, miR-19a, miR-19b-1, miR-20a, miR-21, miR-29a/b/c, miR-30b, miR-31, miR-34a, miR-92a-1, miR-106a, miR-125a/b, miR-142-3p, miR-146a, miR-150, miR-155, miR-181a, miR-223 and miR-424, miR-221, miR-222, let-7i, miR-148, and miR-152.

The isolated nucleic acids of the disclosure may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs). The transgene may comprise a region encoding, for example, a protein and/or an expression control sequence (e.g., a poly-A tail), as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the disclosure is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the isolated nucleic acid further comprises a region (e.g., a second region, a third region, a fourth region, etc.) comprising a second AAV ITR. In some embodiments, an isolated nucleic acid encoding a transgene is flanked by AAV ITRs (e.g., in the orientation 5′-ITR-transgene-ITR-3′). In some embodiments, the AAV ITRs are AAV2 ITRs. In some embodiments, at least one of the AAV ITRs is a AITR, which lacks a terminal resolution site and induces formation of a self-complementary AAV (scAAV) vector.

Vectors

Aspects of the disclosure relate to vectors comprising an isolated nucleic acid encoding RGNs and/or PTM expression cassettes. As used herein, the term “vector” includes any genetic element, such as a plasmid, phage, transposon, cosmid, chromosome, artificial chromosome, virus (e.g., AAV as described elsewhere herein), virion, etc., which is capable of replication when associated with the proper control elements and which can transfer gene sequences between cells. In some embodiments, a vector is a viral vector, such as an rAAV vector, a lentiviral vector, an adenoviral vector, a retroviral vector, etc. Thus, the term includes cloning and expression vehicles, as well as viral vectors. In some embodiments, useful vectors are contemplated to be those vectors in which the nucleic acid segment to be transcribed is positioned under the transcriptional control of a promoter.

In some aspects, the disclosure provides isolated adeno-associated viruses (AAVs). As used herein with respect to AAVs, the term “isolated” refers to an AAV that has been artificially produced or obtained. Isolated AAVs may be produced using recombinant methods. Such AAVs are referred to herein as “recombinant AAVs.” Recombinant AAVs (rAAVs) preferably have tissue-specific targeting capabilities, such that a transgene of the rAAV will be delivered specifically to one or more predetermined tissue(s) (e.g., ocular tissues, neurons). The AAV capsid is an important element in determining these tissue-specific targeting capabilities (e.g., tissue tropism). Thus, an rAAV having a capsid appropriate for the tissue being targeted can be selected.

Methods for obtaining recombinant AAVs having a desired capsid protein are well known in the art. (See, for example, US 2003/0138772), the contents of which are incorporated herein by reference in their entirety). Typically the methods involve culturing a host cell which contains a nucleic acid sequence encoding an AAV capsid protein; a functional rep gene; a recombinant AAV vector composed of AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2, and VP3), all of which are transcribed from a single cap gene via alternative splicing. In some embodiments, the molecular weights of VP1, VP2, and VP3 are respectively about 87 kDa, about 72 kDa, and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV (e.g., the AAV, rAAVs of the present disclosure) capsid protein has a tropism for liver tissue (e.g., hepatocytes, etc.). In some embodiments, an AAV capsid protein does not target neuronal cells. In some embodiments, an AAV capsid protein targets neuronal cells. In some embodiments, an AAV capsid protein does not cross the blood-brain barrier (BBB). In some embodiments, an AAV capsid protein crosses the blood-brain barrier (BBB).

In some embodiments, an AAV capsid protein is of an AAV serotype selected from the group consisting of AAV1, AAV2, AAV3, AAV4, AAV5, AAV6, AAV7, AAV8, AAV9, AAV9.hr, AAVrh8, AAVrh10, AAVrh39, AAVrh43, AAV.PHP.B, AAV.PHP.eB, and variants of any of the foregoing. In some embodiments, an AAV capsid protein is of a serotype derived from a non-human primate, for example AAVrh8 serotype.

In some embodiments, an rAAV vector or rAAV particle comprises a mutant ITR that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ΔTRS ITR). Without wishing to be bound by any particular theory, a rAAV vector comprising an ITR lacking a functional TRS produces a self-complementary rAAV vector, for example as described by McCarthy (2008) Molecular Therapy 16(10):1648-1656.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

In some embodiments, the disclosure relates to a host cell containing a nucleic acid that comprises a coding sequence encoding a transgene (e.g., an RGN and/or a PTM expression cassette). A “host cell” refers to any cell that harbors, or is capable of harboring, a substance of interest. Often a host cell is a mammalian cell. In some embodiments, a host cell is a neuron. In some embodiments, a host cell is a photoreceptor cell. In some embodiments, a host cell is a muscle cell. In some embodiments, a host cell is a liver cell. In some embodiments, a host cell is a kidney cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation. In some embodiments, the host cell is a mammalian cell, a yeast cell, a bacterial cell, an insect cell, a plant cell, or a fungal cell. In some embodiments, the host cell is a hepatocyte.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the disclosure. See, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an AAV vector (comprising a transgene flanked by ITR elements) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). Non-limiting examples of vectors suitable for use with the disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpes virus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al., (1973) Virology, 52:456, Sambrook et al., (1989) Molecular Cloning, a laboratory manual, Cold Spring Harbor Laboratories, New York, Davis et al., (1986) Basic Methods in Molecular Biology, Elsevier, and Chu et al., (1981) Gene 13:197. Such techniques can be used to introduce one or more exogenous nucleic acids, such as a nucleotide integration vector and other nucleic acid molecules, into suitable host cells.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

Methods

Methods for delivering a transgene (e.g., an isolated nucleic acid encoding an RGN and/or a PTM expression cassette) to a subject are provided by the disclosure. The methods typically involve administering to a subject an effective amount of an isolated nucleic acid encoding the transgene(s). In some embodiments, expression constructs described by the disclosure are useful for treating diseases associated with a gene having one or more mutations that result in loss or reduction of protein function or activity. Examples of such diseases include but are not limited to Alpha-1-anyitrypsin deficiency (A1AT deficiency), Duchene's muscular dystrophy (DMD), cystic fibrosis (CF), spinal muscular atrophy (SMA), Rett syndrome, and mucopolysaccharidosis (MPS).

The disclosure is based, in part, on the recognition that delivery of PTMs and RNA-guided nucleases resulting in exon replacement facilitates treatment of diseases where gene replacement by AAV-based delivery is limited due to the size of such genes exceeding the packaging capacity (˜4.5-5.5 kb) of AAV genomes. Examples of genes that exceed the packaging capacity of a single rAAV genome include but are not limited to dystrophin (DMD, associated with Duchene muscular dystrophy), dysferlin (DYSF, associated with Miyoshi myopathy, Limb-girdle dystrophy, and distal myopathy), Cystic fibrosis transmembrane conductance regulator (CFTR, associated with cystic fibrosis), myosin VIIA (MYO7A, associated with Usher syndrome), ATP-binding cassette, sub-family A (ABC1), member 4 (ABCA4, associated with Stargardt disease), and Factor VIII (FVIII, associated with hemophilia A). In some embodiments, each of the foregoing diseases comprises one or more mutations in an exon that results in a loss of protein function. In some embodiments, the disclosure relates to delivery of a PTM described herein to mediate replacement of the mutant exon(s) and to restore protein function and/or activity in a gene having the one or more exonic mutations and that exceeds the packaging capacity of a single rAAV genome. Without wishing to be bound by any particular theory, use of certain RGNs (e.g., Cas13 nucleases) which target mRNA and not DNA are advantageous relative to use of other gene editing technologies because they do alter endogenous DNA at the genomic level.

Aspects of the disclosure relate to methods of treating cancer. The disclosure is based, in part, the recognition that delivery of PTMs and RNA-guided nucleases resulting in exon replacement enables correction of aberrant splicing of certain genes involved in cancer (e.g., oncogenes). Correction of aberrant splicing, in some embodiments, switches expression from an oncogenic isoform of a gene to a non-oncogenic (e.g., wild-type or healthy) isoform of the gene. Examples of oncogenic and non-oncogenic isoforms of genes associated with cancer are described, for example by Di et al., (2018) Cell Death and Differentiation, doi:10.1038/s4141802313. In some embodiments, the gene is associated with tumor cell proliferation, metastasis, apoptosis, or resistance to therapeutic modalities (e.g., drug resistance, radiotherapy, etc.). Examples of genes associated with tumor cell proliferation include but are not limited to cyclin D1, Syk, RASSF5, WT1, etc. Examples of genes associated with tumor cell metastasis include but are not limited to CD44, CrkII, KLF6, etc. Examples of genes associated with apoptosis include but are not limited to BCL-Xs, nCLu, ELF2A, RIP3, etc. Examples of genes associated with cancer drug resistance include but are not limited to AR, BRCA1, c-FLIP(L), Survivin, etc. In some embodiments, correction of aberrant splicing of a gene associated with drug resistance restores sensitivity of the cancer cells to one or more therapeutic agents (e.g., cancer drugs). Examples of genes associated with radiotherapy sensitivity include but are not limited to nCLu, Mcl-1L, NPM1, Tap73, etc. In some embodiments, correction of aberrant splicing of a gene associated with radiotherapy sensitivity restores sensitivity of the cancer cells to one or more therapeutic agents (e.g., cancer drugs).

Aspects of the disclosure relate to compositions and methods for treating a subject that has a dominantly inherited disease. As used herein, a “dominantly inherited disease” refers to a disease caused by a mutation in one allele of a gene of a subject that causes altered protein expression or activity in the subject. A dominantly inherited disease may cause a loss of protein function or activity (e.g., haploinsufficency), expression of a dominant negative gene product (e.g., a dominant negative protein). In some embodiments, a loss of protein function or activity is caused by a nonsense mutation (e.g., a mutation that introduces one or more premature stop codons) in the gene. In some embodiments, a dominantly inherited disease may cause a gain of function mutation. In some embodiments, the gain of function results in a new activity of the expressed protein resulting in a disease or disorder. Examples of dominantly inherited diseases include but are not limited to Huntington's disease, retinitis pigmentosa (RP), osteogenesis imperfecta, myotonic dystrophy, spinocerebellar ataxia 3, frontotemporal dementia (FTD), neurofibranatosis (type 1 or type 2), Marfan syndrome, Von Willebrand disease, familial hypercholesterolemia, tuberous sclerosis, amyotrophic lateral sclerosis (ALS), and Autosomal Dominant Polycystic Kidney Disease (ADPKD). Dominantly inherited diseases, in some embodiments, are caused by one or more mutations in a protein encoded by a gene, for example HTT, RP1, RP2, COL1A1, COL1A2, DMPK, NF1, FBN1, VWF, TSC1, TSC2, SOD1, PKD1, PKD2, etc.

Aspects of the disclosure relate to compositions and methods for treating a subject that has a recessively inherited disease. As used herein, a “recessively inherited disease” refers to a disease caused by a mutation or mutations in both alleles of a gene of a subject that causes altered protein expression or activity in the subject. A recessively inherited disease may cause a loss of protein function or activity. In some embodiments, a loss of protein function or activity is caused by a nonsense mutation (e.g., a mutation that introduces one or more premature stop codons) in both alleles of the gene. Examples of recessively inherited diseases include but are not limited to cystic fibrosis (CF), Fanconi anemia, Pyruvate Dehydrogenase Deficiency, Pompe's disease, Gaucher's disease, phenylketonuria (PKU), and maple syrup urine disease (MSUD). Recessively inherited diseases, in some embodiments, are caused by one or more mutations in a protein encoded by a gene, for example, CFTR, FANCA, PDHA1, GAA, GBA1, PAH, BCKDHA, etc.

The disclosure is based, in part, on Cas13 nucleases (and variants thereof) which are configured to mediate trans-splicing of a donor sequence (e.g., a donor exon) in order to replace a mutant exon (e.g., an exon of a gene of interest having one or more mutations, substitutions, insertions, deletions, etc. relative to the corresponding wild-type exon of the gene of interest). In some embodiments, spliceosome-mediated RNA trans-splicing modifies mRNA by replacing a portion of the target endogenous pre-mRNA sequence (e.g., a mutant exon of a gene of interest) with exogenous RNA (e.g., a non-mutant exon, as delivered by a PTM) in a trans-splicing event. In some embodiments, trans-splicing editing repairs a broader range of genetic mutations, including not only point mutations, but also indels or other complicated mutations, relative to other gene therapy technologies. The repair mRNA product is subjected to the endogenous transcriptional regulation of the cell, which is an advantage. The reduction of delivered gene size also makes it more feasible for viral vector delivery.

Accordingly, in some aspects, the disclosure provides a method for replacing a mutant exon of a gene of interest in a cell, the method comprising expressing in a cell having a mutant exon of a gene of interest: an isolated nucleic acid encoding a PTM as described herein, wherein one or more of the gRNAs encoded by the isolated nucleic acid specifically bind to an intron-exon boundary that is 5′ relative to the mutant exon of the gene of interest; and an RNA-guided nuclease, wherein the donor sequence of the isolated nucleic acid encodes a wild-type exon of the gene of interest corresponding to the mutant exon.

In some aspects, the disclosure provides a method for treating a disease associated with a loss of protein function in a subject in need thereof, the method comprising administering to the subject: an isolated nucleic acid encoding a PTM, wherein one or more of the gRNAs encoded by the isolated nucleic acid specifically bind to an intron-exon boundary that is 5′ relative to a mutant exon of a gene of interest; and an RNA-guided nuclease, wherein the subject has a disease characterized by the presence of the mutant exon in the gene of interest, and wherein the donor sequence of the isolated nucleic acid encodes a wild-type exon of the gene of interest corresponding to the mutant exon.

The term “subject,” as used herein, refers to any organism in need of treatment or diagnosis using the subject matter herein. For example, without limitation, subjects may include mammals and non-mammals. In some embodiments, a subject is mammalian. In some embodiments, a subject is non-mammalian. As used herein, a “mammal,” refers to any animal constituting the class Mammalia (e.g., a human, mouse, rat, cat, dog, sheep, rabbit, horse, cow, goat, pig, guinea pig, hamster, chicken, turkey, or a non-human primate (e.g., Marmoset, Macaque)). In some embodiments, a mammal is a human.

An “effective amount” of a substance is an amount sufficient to produce a desired effect. In some embodiments, an effective amount of an isolated nucleic acid is an amount sufficient to transfect (or infect in the context of rAAV mediated delivery) a sufficient number of target cells of a target tissue of a subject. In some embodiments, an effective amount of an isolated nucleic acid (e.g., which may be delivered via an rAAV) may be an amount sufficient to have a therapeutic benefit in a subject, e.g., to increase the expression or activity of one or more proteins (e.g., to restore function in a loss of function disease), to extend the lifespan of a subject, to improve in the subject one or more symptoms of disease, etc. The effective amount will depend on a variety of factors such as, for example, the species, age, weight, health of the subject, and the tissue to be targeted, and may thus vary among subject and tissue as described elsewhere in the disclosure.

In some embodiments, expression of an isolated nucleic acid and an RNA-guided nuclease results in translation of a full-length, wild-type gene product of a gene of interest. In some embodiments, translation of the full-length, wild-type gene product results in a protein activity increase in the cell. In some embodiments, the increase in protein activity in the cell ranges from about 1% to about 1,000% (e.g., relative to a cell having a copy of the gene of interest with a mutant exon that has not been replaced). In some embodiments, the increase in protein activity in the cell ranges from about 0.5-fold to about 1,000-fold (e.g., relative to a cell having a copy of the gene of interest with a mutant exon that has not been replaced).

As used herein, the terms “treat” or “treating,” depending on context and tense, refer to the application or administration of a composition encoding a transgene(s) to a subject, who has a disease characterized by a loss or reduction of protein function in a particular gene, or a predisposition toward a disease characterized by a loss or reduction of protein function in a particular gene, with the purpose to cure, heal, alleviate, relieve, alter, remedy, ameliorate, improve, or affect the disorder, the symptom of the disease, or the predisposition toward the disease.

Alleviating a disease includes delaying the development or progression of the disease, or reducing disease severity. Alleviating the disease does not necessarily require curative results. As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a disease includes initial onset and/or recurrence.

In some embodiments, administration occurs via systemic injection or direct injection to the liver. In some embodiments, systemic injection is intravenous injection. In some embodiments, direct injection is intravenous injection, intracerebral injection, intraparenchymal injection, intrahepatic injection (e.g., hepatic portal vein injection, etc.), or intraocular injection.

Administration

The isolated nucleic acids and rAAVs of the disclosure may be delivered to a subject in compositions according to any appropriate methods known in the art. For example, a composition (e.g., an isolated nucleic acid or rAAV), preferably suspended in a physiologically compatible carrier (i.e., in a composition), may be administered to a subject, i.e., host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human.

Delivery of the compositions (e.g., isolated nucleic acids, rAAVs, etc.) to a mammalian subject may be by, for example, intramuscular injection or by administration into the bloodstream of the mammalian subject. Administration into the bloodstream may be by injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue.

The compositions of the disclosure may comprise an rAAV alone, or in combination with one or more other viruses (e.g., a second rAAV encoding having one or more different transgenes, a different type of viral vector such as a lentiviral vector having one or more different transgenes, etc.). In some embodiments, a composition comprises 1, 2, 3, 4, 5, 6, 7, 8, 9, 10, or more different viral vectors (e.g., rAAVs, etc.) each having one or more different transgenes.

Suitable carriers may be readily selected by one of skill in the art in view of the indication for which the composition is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the compositions and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The compositions (e.g., isolated nucleic acids, rAAVs, etc.) are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intraocular, intravenous, intracerebroventricular, intramuscular, subcutaneous, intradermal, intratumoral, and other parental routes of administration. Routes of administration may be combined, if desired.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art.

Consistent with the definition of “effective amount” as discussed elsewhere herein, an effective amount of an rAAV is an amount sufficient to target infect an animal, target a desired tissue. In some embodiments, an effective amount of an rAAV is an amount sufficient to produce a stable somatic transgenic animal model. The effective amount will depend primarily on factors such as the species, age, weight, health of the subject, and the tissue to be targeted, and may thus vary among animal and tissue. For example, an effective amount of the rAAV is generally in the range of from about 1 ml to about 100 ml of solution containing from about 10⁹ to 10¹⁶ genome copies. In some cases, a dosage between about 10¹¹ to 10¹³ rAAV genome copies is appropriate. In certain embodiments, 10¹² or 10¹³ rAAV genome copies is effective to target CNS tissue. In some cases, stable transgenic animals are produced by multiple doses of an rAAV.

In some embodiments, a dose of a composition (e.g., isolated nucleic acid, rAAV, etc.) is administered to a subject no more than once per calendar day (e.g., a 24-hour period). In some embodiments, a dose of rAAV is administered to a subject no more than once per 2, 3, 4, 5, 6, or 7 calendar days. In some embodiments, a dose of rAAV is administered to a subject no more than once per calendar week (e.g., 7 calendar days). In some embodiments, a dose of rAAV is administered to a subject no more than bi-weekly (e.g., once in a two calendar week period). In some embodiments, a dose of rAAV is administered to a subject no more than once per calendar month (e.g., once in 30 calendar days). In some embodiments, a dose of rAAV is administered to a subject no more than once per six calendar months. In some embodiments, a dose of rAAV is administered to a subject no more than once per calendar year (e.g., 365 days or 366 days in a leap year).

In some embodiments, rAAV compositions are formulated to reduce aggregation of AAV particles in the composition, particularly where high rAAV concentrations are present (e.g., ˜10¹³ GC/ml or more). Methods for reducing aggregation of rAAVs are well known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either subcutaneously, intraopancreatically, intranasally, parenterally, intravenously, intramuscularly, intrathecally, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

### EXAMPLES

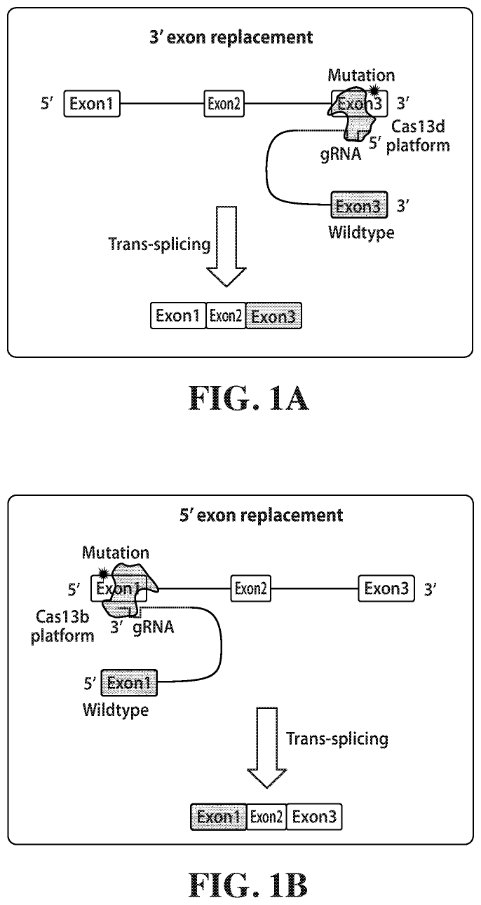

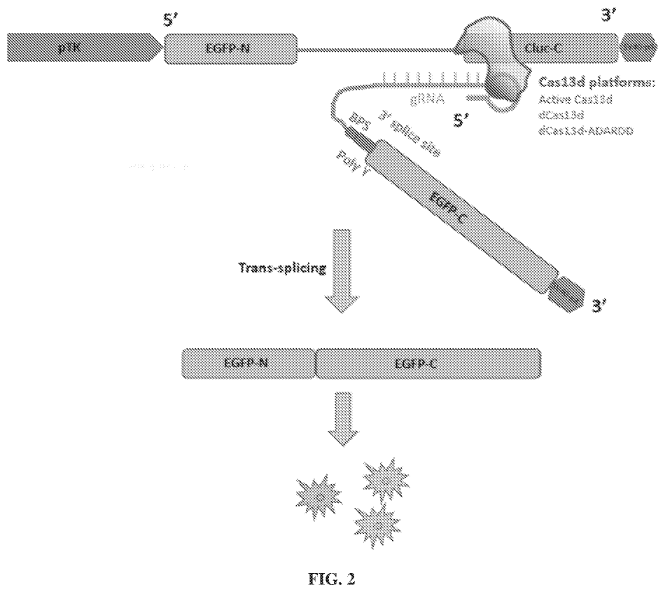

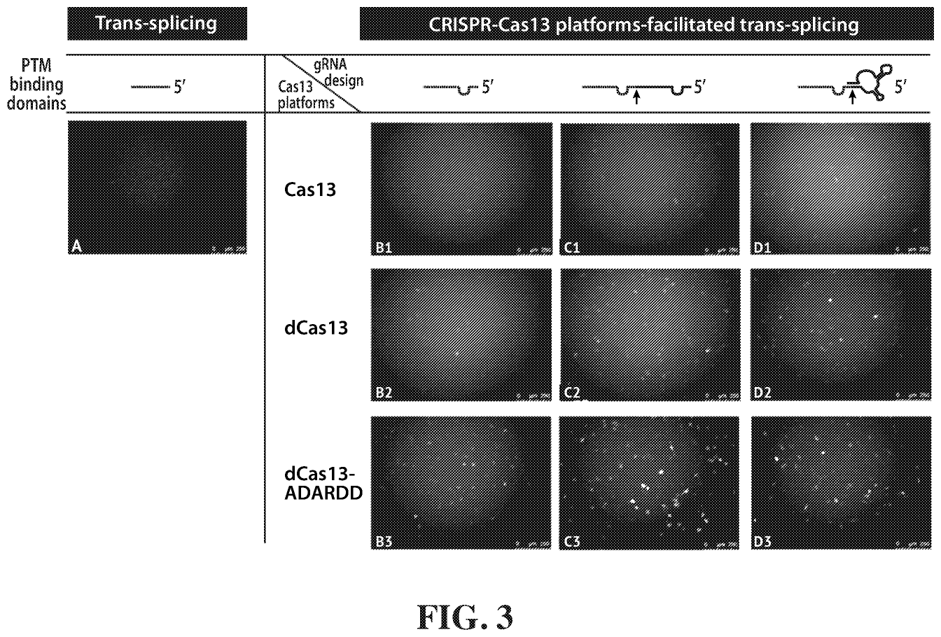

This example describes a CRISPR-Cas13-facilitated RNA trans-splicing therapy strategy. The RNA targeting and manipulation abilities of CRISPR-Cas13 are applied to the spliceosome-mediated RNA trans-splicing process to improve the specificity and efficiency. First, the pre-trans-splicing molecule (PTM) was fused with the gRNA of Cas13; the two parts of RNA sequence are transcribed as one, processed as one, and targeted as one. For 3′ exon replacement, the gRNA locates at the 5′ of the combined PTM (FIG. 1A). For 5′ exon replacement, the gRNA locates at the 3′ of the fusion PTM (FIG. 1B). Since the gRNA and PTM are transcribed by Pol II RNA polymerase, the 5′ GpppG and 3′ polyA modifications may affect the targeting efficiency of Cas13-gRNA. The unwanted modifications were removed by two methods, leveraging the gRNA cleavage ability of Cas13 or adding a self-cleaving hammerhead ribozyme. The RNA targeting ability of Cas13 gRNA could efficiently bring the PTM to the endogenous RNA. Second, different orthologues and versions of Cas13 proteins are used according to particular replacement strategy-Cas13d for 3′ exon replacement and Cas13b for 5′ exon replacement. Both active and dead versions of Cas13 proteins are used to cut or mask the endogenous RNA sequence, respectively. Besides the Cas13 protein, a fusion of dCas13 with adenosine deaminase acting on RNA deaminase domain (ADARDD) is also included to change the critical adenosine (A) as splicing branch point to hypoxanthine (I). All of these three kinds of Cas13 proteins are designed to inhibit the cis-splicing event, making the trans-splicing more efficient. The CRISPR-Cas13-facilitated RNA trans-splicing efficiency was tested in HEK 293 cells with a reporter (FIG. 2). In this reporter system, the N terminal and C terminal halves of EGFP are separately transcribed. Trans-splicing reconstitutes the full-length EGFP mRNA and results in EGFP fluorescence. Based on the preliminary data (FIG. 3), it was concluded that CRISPR-Cas13 platforms could efficiently facilitate RNA trans-splicing.

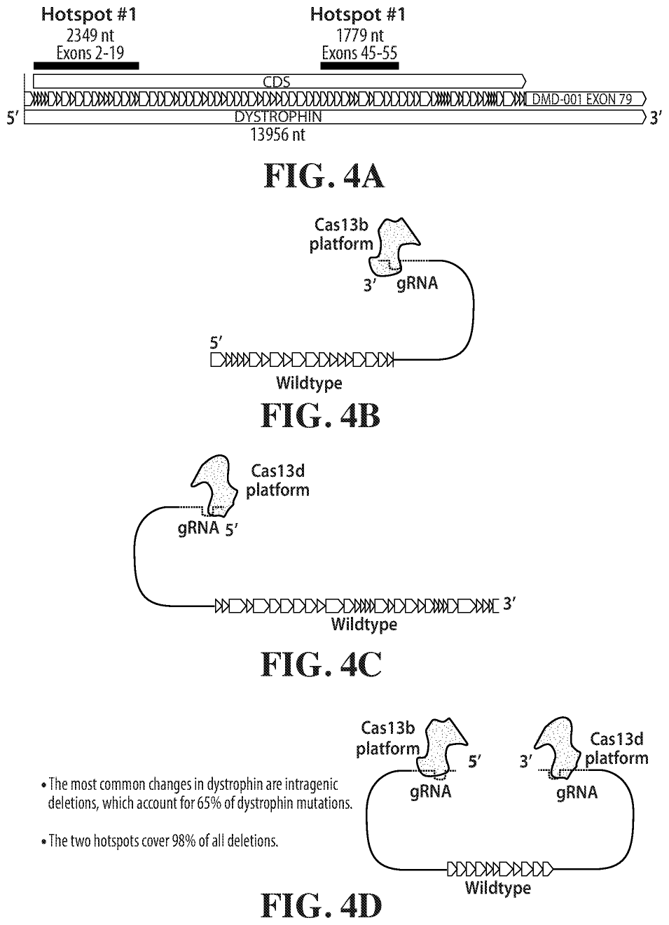

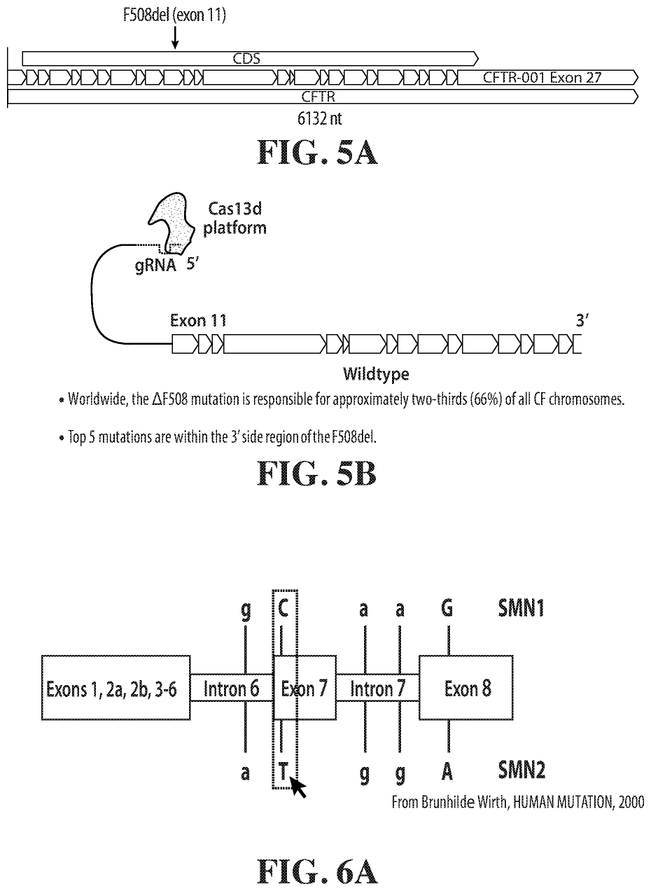

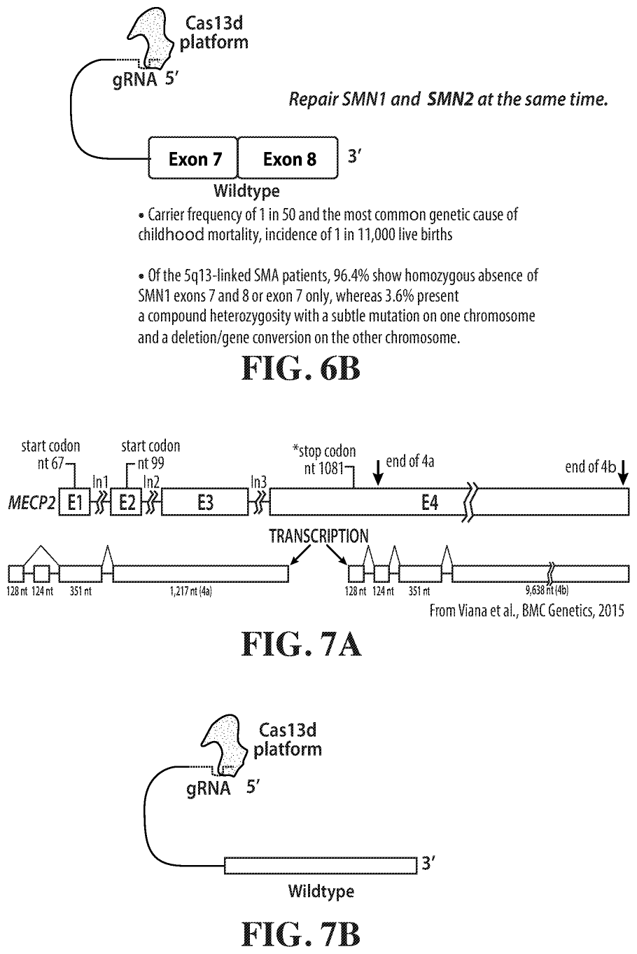

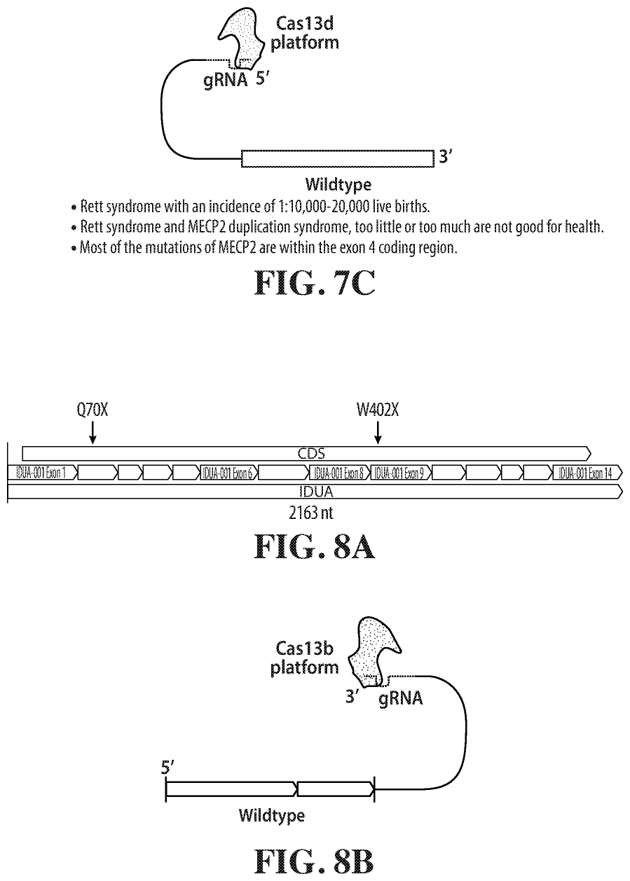

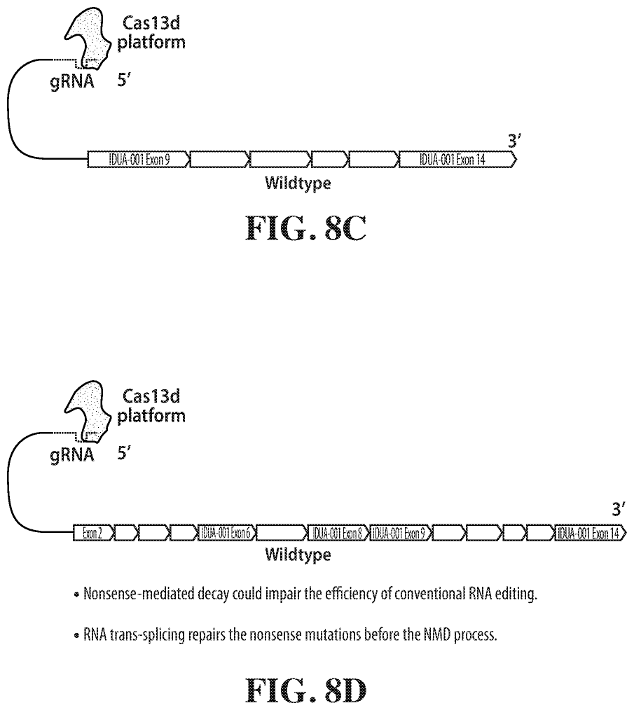

FIGS. 4A-8D show schematics depicting gene therapy by CRISPR-Cas13-facilitated RNA Trans-splicing for several representative diseases, including Duchene's muscular dystrophy (DMD), cystic fibrosis (CF), spinal muscular atrophy (SMA), Rett syndrome, and mucopolysaccharidosis (MPS).

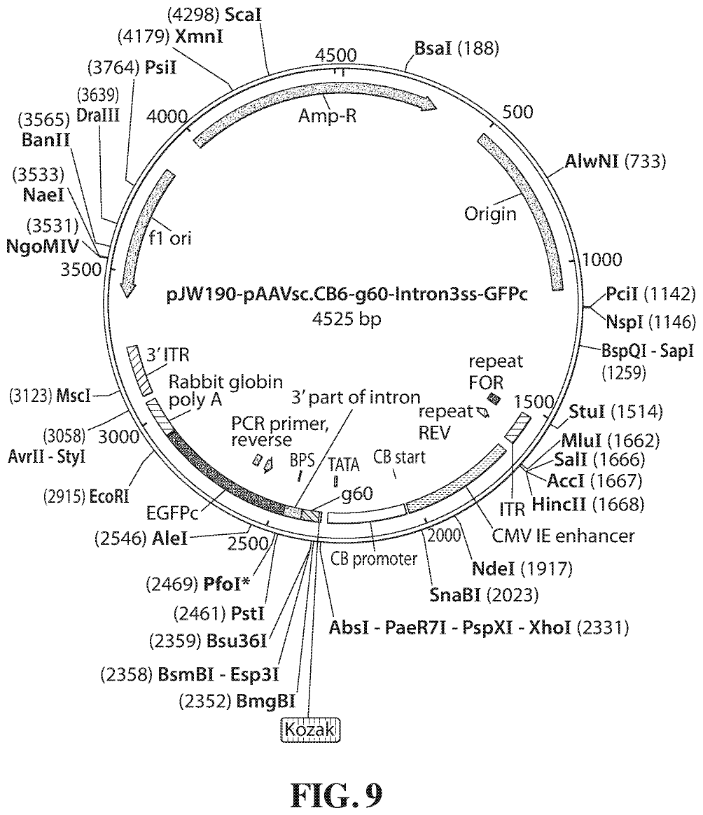

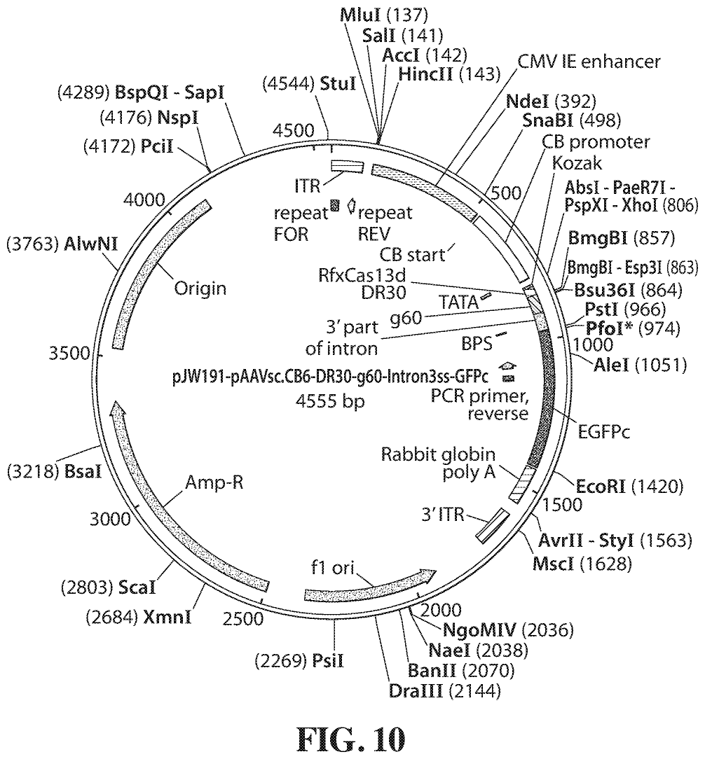

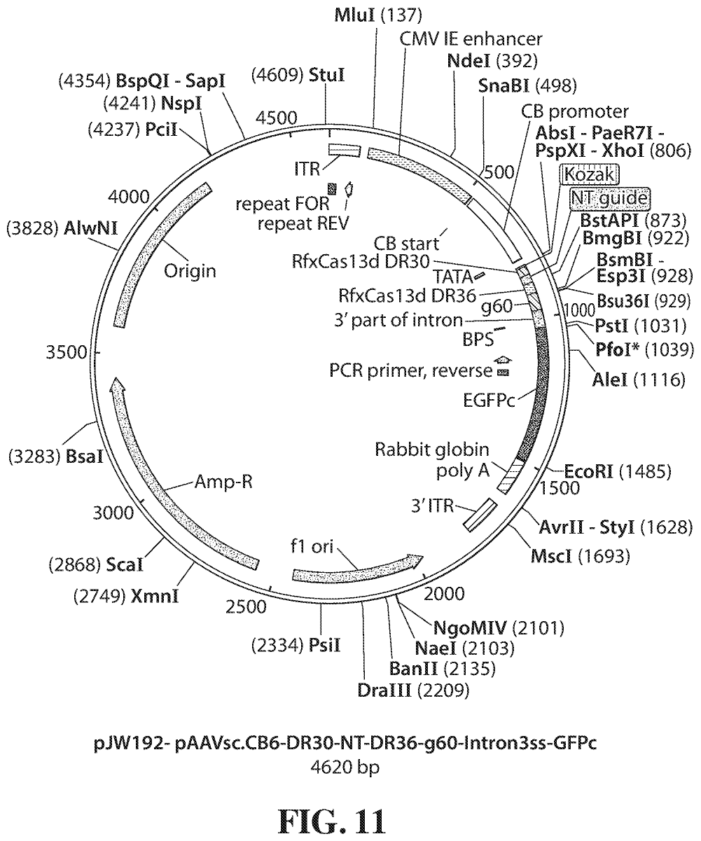

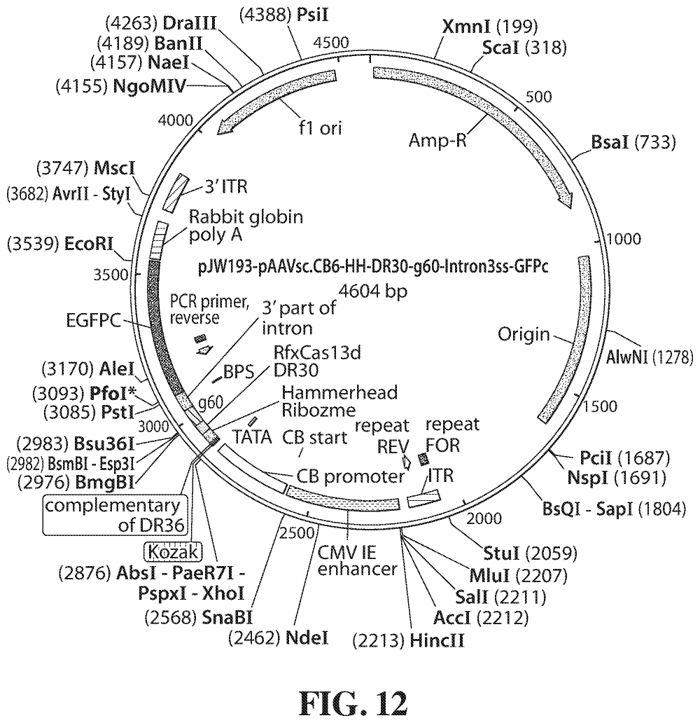

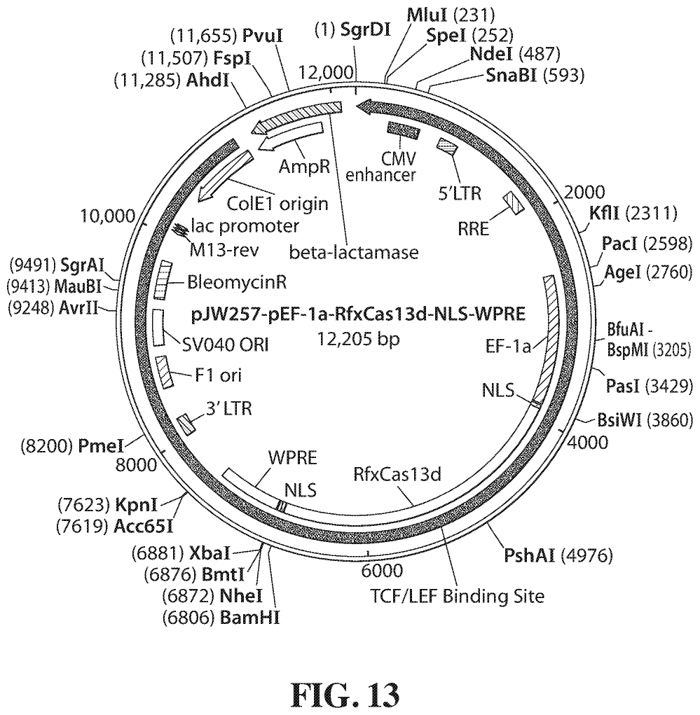

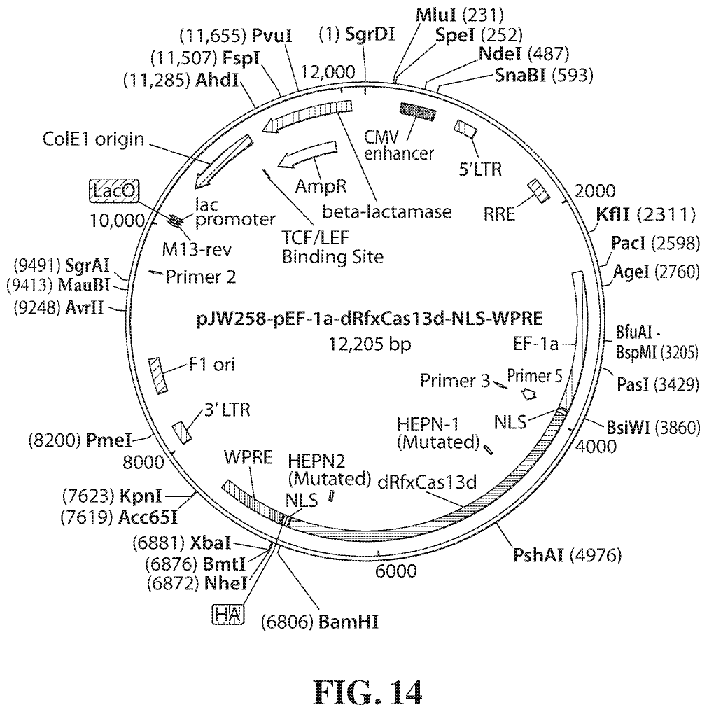

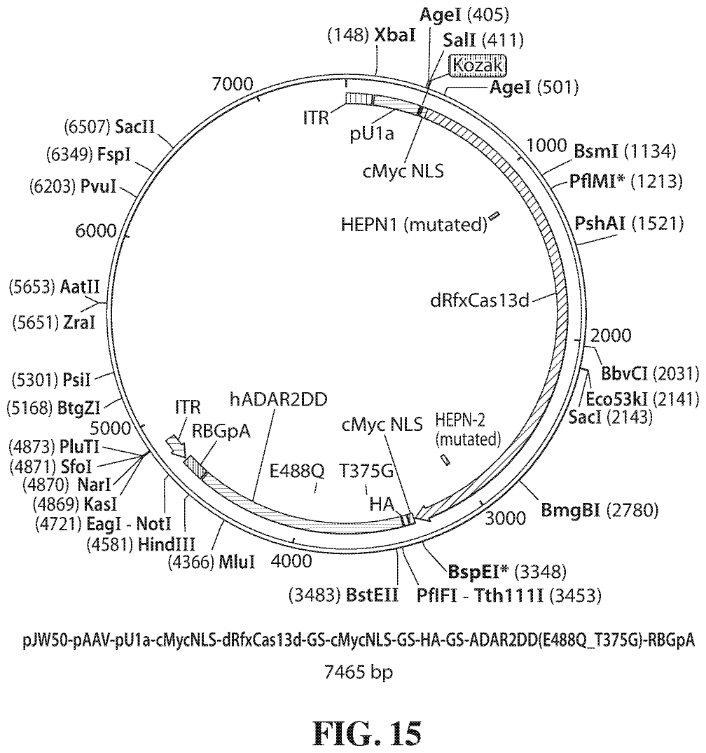

FIGS. 9-15 show vector maps depicting non-limiting examples of plasmids encoding PTS molecules and/or RNA-guided nucleases.

### Exemplary Sequences

The following are some exemplary sequences as disclosed by the instant Specification, but are not limiting. This Specification includes a Sequence Listing submitted concurrently herewith as a text file in ASCII format. The Sequence Listing and all of the information contained therein are expressly incorporated herein and constitute part of the instant Specification as filed.

In some embodiments, an isolated nucleic acid comprises or consists of one of the following sequences, or the reverse complement of one of the following sequences. In some embodiments, an isolated nucleic acid comprises or consists of a portion of one of the following sequences (e.g., a gRNA portion, an intronic sequence portion, a donor sequence portion, an rAAV vector portion, etc.), or the reverse complement of a portion of one of the following sequences.

>pJW190-pAAVsc.CB6-g60-Intron3ss-GFPc;
SEQ ID NO: 1
ctgcgcgctcgctcgctcactgaggccgcccgggcaaagcccgggcgtcgggcga
cctttggtcgcccggcctcagtgagcgagcgagcgcgcagagagggagtgtagcc
atgctctaggaagatcaattcaattcacgcgtcgacattgattattgactagtta
ttaatagtaatcaattacggggtcattagttcatagcccatatatggagttccgc
gttacataacttacggtaaatggcccgcctggctgaccgcccaacgacccccgcc
cattgacgtcaataatgacgtatgttcccatagtaacgccaatagggactttcca
ttgacgtcaatgggtggatatttacggtaaactgcccacttggcagtacatcaag
tgtatcatatgccaagtacgccccctattgacgtcaatgacggtaaatggcccgc
ctggcattatgcccagtacatgaccttatgggactttcctacttggcagtacatc
tacgtattagtcatcgctattaccatgtcgaggccacgttctgcttcactctccc
tgtgcagcgatgggggcggggggggggggcgcgcgccaggcggggcggggcgggg
cgaggggcggggcggggcgaggcggagaggtgcggcggcagccaatcagagcggc
gcgctccgaaagtttccttttatggcgaggcggcggcggcggcggccctataaaa
agcgaagcgcgcggcgggcgggagcaagctctagcctcgaggccaccAGCAGCGC
ACGTCTCCCTAAGGGTGGGAAAATAGACCAATAGGCAGAGAGAGTCAGTGCCgga
tcccagcgatatcaaaactagtgtgacgctgctgacccctttctttcccttctgc
agggtacgtccaggagcgcaccatcttcttcaaggacgacggcaactacaagacc
cgcgccgaggtgaagttcgagggcgacaccctggtgaaccgcatcgagctgaagg
gcatcgacttcaaggaggacggcaacatcctggggcacaagctggagtacaacta
caacagccacaacgtctatatcatggccgacaagcagaagaacggcatcaaggcg
aacttcaagatccgccacaacatcgaggacggcggcgtgcagctcgccgaccact
accagcagaacacccccatcggcgacggccccgtgctgctgcccgacaaccacta
cctgagcacccagtccgccctgagcaaagaccccaacgagaagcgcgatcacatg
gtcctgctggagttcgtgaccgccgccgggatcactctcggcatggacgagcttt
acaaggatatccctgaattctagggatccgatctttttccctctgccaaaaatta
tggggacatcatgaagccccttgagcatctgacttctggctaataaaggaaattt
attttcattgcaatagtgtgttggaattttttgtgtctctcactcggcctaggta
gataagtagcatggcgggttaatcattaactacaaggaacccctagtgatggagt
tggccactccctctctgcgcgctcgctcgctcactgaggccgggcgaccaaaggt
cgcccgacgcccgggctttgcccgggcggcctcagtgagcgagcgagcgcgcag
>pJW191-pAAVsc.CB6-DR30-g60-Intron3ss-GFPc;
SEQ ID NO: 2
ctgcgcgctcgctcgctcactgaggccgcccgggcaaagcccgggcgtcgggcga
cctttggtcgcccggcctcagtgagcgagcgagcgcgcagagagggagtgtagcc
atgctctaggaagatcaattcaattcacgcgtcgacattgattattgactagtta
ttaatagtaatcaattacggggtcattagttcatagcccatatatggagttccgc
gttacataacttacggtaaatggcccgcctggctgaccgcccaacgacccccgcc
cattgacgtcaataatgacgtatgttcccatagtaacgccaatagggactttcca
ttgacgtcaatgggtggatatttacggtaaactgcccacttggcagtacatcaag
tgtatcatatgccaagtacgccccctattgacgtcaatgacggtaaatggcccgc
ctggcattatgcccagtacatgaccttatgggactttcctacttggcagtacatc
tacgtattagtcatcgctattaccatgtcgaggccacgttctgcttcactctccc
tgtgcagcgatgggggcggggggggggggcgcgcgccaggcggggcggggcgggg
cgaggggcggggcggggcgaggcggagaggtgcggcggcagccaatcagagcggc
gcgctccgaaagtttccttttatggcgaggcggcggcggcggcggccctataaaa
agcgaagcgcgcggcgggcgggagcaagctctagcctcgaggccaccaaccccta
ccaactggtcggggtttgaaacAGCAGCGCACGTCTCCCTAAGGGTGGGAAAATA
GACCAATAGGCAGAGAGAGTCAGTGCCggatcccagcgatatcaaaactagtgtg
acgctgctgacccctttctttcccttctgcagggtacgtccaggagcgcaccatc
ttcttcaaggacgacggcaactacaagacccgcgccgaggtgaagttcgagggcg
acaccctggtgaaccgcatcgagctgaagggcatcgacttcaaggaggacggcaa
catcctggggcacaagctggagtacaactacaacagccacaacgtctatatcatg
gccgacaagcagaagaacggcatcaaggcgaacttcaagatccgccacaacatcg
aggacggcggcgtgcagctcgccgaccactaccagcagaacacccccatcggcga
cggccccgtgctgctgcccgacaaccactacctgagcacccagtccgccctgagc
aaagaccccaacgagaagcgcgatcacatggtcctgctggagttcgtgaccgccg
ccgggatcactctcggcatggacgagctttacaaggatatccctgaattctaggg
atccgatctttttccctctgccaaaaattatggggacatcatgaagccccttgag
catctgacttctggctaataaaggaaatttattttcattgcaatagtgtgttgga
attttttgtgtctctcactcggcctaggtagataagtagcatggcgggttaatca
ttaactacaaggaacccctagtgatggagttggccactccctctctgcgcgctcg
ctcgctcactgaggccgggcgaccaaaggtcgcccgacgcccgggctttgcccgg
gcggcctcagtgagcgagcgagcgcgcag
>pJW192-pAAVsc.CB6-DR30-NT-DR36-g60-Intron3ss-GFPc;
SEQ ID NO: 3
ctgcgcgctcgctcgctcactgaggccgcccgggcaaagcccgggcgtcgggcga
cctttggtcgcccggcctcagtgagcgagcgagcgcgcagagagggagtgtagcc
atgctctaggaagatcaattcaattcacgcgtcgacattgattattgactagtta
ttaatagtaatcaattacggggtcattagttcatagcccatatatggagttccgc
gttacataacttacggtaaatggcccgcctggctgaccgcccaacgacccccgcc
cattgacgtcaataatgacgtatgttcccatagtaacgccaatagggactttcca
ttgacgtcaatgggtggatatttacggtaaactgcccacttggcagtacatcaag
tgtatcatatgccaagtacgccccctattgacgtcaatgacggtaaatggcccgc
ctggcattatgcccagtacatgaccttatgggactttcctacttggcagtacatc
tacgtattagtcatcgctattaccatgtcgaggccacgttctgcttcactctccc
tgtgcagcgatgggggcggggggggggggcgcgcgccaggcggggcggggcgggg
cgaggggcggggcggggcgaggcggagaggtgcggcggcagccaatcagagcggc
gcgctccgaaagtttccttttatggcgaggcggcggcggcggcggccctataaaa
agcgaagcgcgcggcgggcgggagcaagctctagcctcgaggccaccaaccccta
ccaactggtcggggtttgaaactaatgcctggcttgtcgacgcatagtctgCAAG
TAAACCCCTACCAACTGGTCGGGGTTTGAAACAGCAGCGCACGTCTCCCTAAGGG
TGGGAAAATAGACCAATAGGCAGAGAGAGTCAGTGCCggatcccagcgatatcaa
aactagtgtgacgctgctgacccctttctttcccttctgcagggtacgtccagga
gcgcaccatcttcttcaaggacgacggcaactacaagacccgcgccgaggtgaag
ttcgagggcgacaccctggtgaaccgcatcgagctgaagggcatcgacttcaagg
aggacggcaacatcctggggcacaagctggagtacaactacaacagccacaacgt
ctatatcatggccgacaagcagaagaacggcatcaaggcgaacttcaagatccgc
cacaacatcgaggacggcggcgtgcagctcgccgaccactaccagcagaacaccc
ccatcggcgacggccccgtgctgctgcccgacaaccactacctgagcacccagtc
cgccctgagcaaagaccccaacgagaagcgcgatcacatggtcctgctggagttc
gtgaccgccgccgggatcactctcggcatggacgagctttacaaggatatccctg
aattctagggatccgatctttttccctctgccaaaaattatggggacatcatgaa
gccccttgagcatctgacttctggctaataaaggaaatttattttcattgcaata
gtgtgttggaattttttgtgtctctcactcggcctaggtagataagtagcatggc
gggttaatcattaactacaaggaacccctagtgatggagttggccactccctctc
tgcgcgctcgctcgctcactgaggccgggcgaccaaaggtcgcccgacgcccggg
ctttgcccgggcggcctcagtgagcgagcgagcgcgcag
>pJW193-pAAVsc.CB6-HH-DR30-g60-Intron3ss-GFPc;
SEQ ID NO: 4
ctgcgcgctcgctcgctcactgaggccgcccgggcaaagcccgggcgtcgggcga
cctttggtcgcccggcctcagtgagcgagcgagcgcgcagagagggagtgtagcc
atgctctaggaagatcaattcaattcacgcgtcgacattgattattgactagtta
ttaatagtaatcaattacggggtcattagttcatagcccatatatggagttccgc
gttacataacttacggtaaatggcccgcctggctgaccgcccaacgacccccgcc
cattgacgtcaataatgacgtatgttcccatagtaacgccaatagggactttcca
ttgacgtcaatgggtggatatttacggtaaactgcccacttggcagtacatcaag
tgtatcatatgccaagtacgccccctattgacgtcaatgacggtaaatggcccgc
ctggcattatgcccagtacatgaccttatgggactttcctacttggcagtacatc
tacgtattagtcatcgctattaccatgtcgaggccacgttctgcttcactctccc
tgtgcagcgatgggggcggggggggggggcgcgcgccaggcggggcggggcgggg
cgaggggcggggcggggcgaggcggagaggtgcggcggcagccaatcagagcggc
gcgctccgaaagtttccttttatggcgaggcggcggcggcggcggccctataaaa
agcgaagcgcgcggcgggcgggagcaagctctagcctcgaggccaccTACTTGCT
GATGAGTCCGTGAGGACGAAACGAGTAAGCTCGTCCAAGTAAACCCCTACCAACT
GGTCGGGGTTTGAAACAGCAGCGCACGTCTCCCTAAGGGTGGGAAAATAGACCAA
TAGGCAGAGAGAGTCAGTGCCggatcccagcgatatcaaaactagtgtgacgctg
ctgacccctttctttcccttctgcagggtacgtccaggagcgcaccatcttcttc
aaggacgacggcaactacaagacccgcgccgaggtgaagttcgagggcgacaccc
tggtgaaccgcatcgagctgaagggcatcgacttcaaggaggacggcaacatcct
ggggcacaagctggagtacaactacaacagccacaacgtctatatcatggccgac
aagcagaagaacggcatcaaggcgaacttcaagatccgccacaacatcgaggacg
gcggcgtgcagctcgccgaccactaccagcagaacacccccatcggcgacggccc
cgtgctgctgcccgacaaccactacctgagcacccagtccgccctgagcaaagac
cccaacgagaagcgcgatcacatggtcctgctggagttcgtgaccgccgccggga
tcactctcggcatggacgagctttacaaggatatccctgaattctagggatccga
tctttttccctctgccaaaaattatggggacatcatgaagccccttgagcatctg
acttctggctaataaaggaaatttattttcattgcaatagtgtgttggaattttt
tgtgtctctcactcggcctaggtagataagtagcatggcgggttaatcattaact
acaaggaacccctagtgatggagttggccactccctctctgcgcgctcgctcgct
cactgaggccgggcgaccaaaggtcgcccgacgcccgggctttgcccgggcggcc
tcagtgagcgagcgagcgcgcag
>pJW257-pEF-1a-RfxCas13d-NLS-WPRE;
SEQ ID NO: 5
gggtctctctggttagaccagatctgagcctgggagctctctggctaactaggga
acccactgcttaagcctcaataaagcttgccttgagtgcttcaagtagtgtgtgc
ccgtctgttgtgtgactctggtaactagagatccctcagacccttttagtcagtg
tggaaaatctctagcagtggcgcccgaacagggacttgaaagcgaaagggaaacc
agaggagctctctcgacgcaggactcggcttgctgaagcgcgcacggcaagaggc
gaggggcggcgactggtgagtacgccaaaaattttgactagcggaggctagaagg
agagagatgggtgcgagagcgtcagtattaagcgggggagaattagatcgcgatg
ggaaaaaattcggttaaggccagggggaaagaaaaaatataaattaaaacatata
gtatgggcaagcagggagctagaacgattcgcagttaatcctggcctgttagaaa
catcagaaggctgtagacaaatactgggacagctacaaccatcccttcagacagg
atcagaagaacttagatcattatataatacagtagcaaccctctattgtgtgcat
caaaggatagagataaaagacaccaaggaagctttagacaagatagaggaagagc
aaaacaaaagtaagaccaccgcacagcaagcggccgctgatcttcagacctggag
gaggagatatgagggacaattggagaagtgaattatataaatataaagtagtaaa
aattgaaccattaggagtagcacccaccaaggcaaagagaagagtggtgcagaga
gaaaaaagagcagtgggaataggagctttgttccttgggttcttgggagcagcag
gaagcactatgggcgcagcgtcaatgacgctgacggtacaggccagacaattatt
gtctggtatagtgcagcagcagaacaatttgctgagggctattgaggcgcaacag
catctgttgcaactcacagtctggggcatcaagcagctccaggcaagaatcctgg
ctgtggaaagatacctaaaggatcaacagctcctggggatttggggttgctctgg
aaaactcatttgcaccactgctgtgccttggaatgctagttggagtaataaatct
ctggaacagatttggaatcacacgacctggatggagtgggacagagaaattaaca
attacacaagcttaatacactccttaattgaagaatcgcaaaaccagcaagaaaa
gaatgaacaagaattattggaattagataaatgggcaagtttgtggaattggttt
aacataacaaattggctgtggtatataaaattattcataatgatagtaggaggct
tggtaggtttaagaatagtttttgctgtactttctatagtgaatagagttaggca
gggatattcaccattatcgtttcagacccacctcccaaccccgaggggacccgac
aggcccgaaggaatagaagaagaaggtggagagagagacagagacagatccattc
gattagtgaacggatcggcactgcgtgcgccaattctgcagacaaatggcagtat
tcatccacaattttaaaagaaaaggggggattggggggtacagtgcaggggaaag
aatagtagacataatagcaacagacatacaaactaaagaattacaaaaacaaatt
acaaaaattcaaaattttcgggtttattacagggacagcagagatccagtttggt
taattaatgcaaagatggataaagttttaaacagagaggaatctttgcagctaat
ggaccttctaggtcttgaaaggagtgggaattggctccggtgcccgtcagtgggc
agagcgcacatcgcccacagtccccgagaagttggggggaggggtcggcaattga
accggtgcctagagaaggtggcgcggggtaaactgggaaagtgatgtcgtgtact
ggctccgcctttttcccgagggtgggggagaaccgtatataagtgcagtagtcgc
cgtgaacgttctttttcgcaacgggtttgccgccagaacacaggtaagtgccgtg
tgtggttcccgcgggcctggcctctttacgggttatggcccttgcgtgccttgaa
ttacttccactggctgcagtacgtgattcttgatcccgagcttcgggttggaagt
gggtgggagagttcgaggccttgcgcttaaggagccccttcgcctcgtgcttgag
ttgaggcctggcctgggcgctggggccgccgcgtgcgaatctggtggcaccttcg
cgcctgtctcgctgctttcgataagtctctagccatttaaaatttttgatgacct
gctgcgacgctttttttctggcaagatagtcttgtaaatgcgggccaagatctgc
acactggtatttcggtttttggggccgcgggcggcgacggggcccgtgcgtccca
gcgcacatgttcggcgaggcggggcctgcgagcgcggccaccgagaatcggacgg
gggtagtctcaagctggccggcctgctctggtgcctggcctcgcgccgccgtgta
tcgccccgccctgggcggcaaggctggcccggteggcaecagttgcgtgagcgga
aagatggccgcttcccggccctgctgcagggagctcaaaatggaggacgcggcgc
tcgggagagcgggcgggtgagtcacccacacaaaggaaaagggcctttccgtcct
cagccgtcgcttcatgtgactccacggagtaccgggcgccgtccaggcacctcga
ttagttctcgagcttttggagtacgtcgtctttaggttggggggaggggttttat
gcgatggagtttccccacactgagtgggtggagactgaagttaggccagcttggc
acttgatgtaattctccttggaatttgccctttttgagtttggatcttggttcat
tctcaagcctcagacagtggttcaaagtttttttcttccatttcaggtgtcgtga
cGTACGgccaccatgAGCCCCAAGAAgAAGAGaAAGGTGGAGGCCAGCATCGAAA
AAAAAAAGTCCTTCGCCAAGGGCATGGGCGTGAAGTCCACACTCGTGTCCGGCTC
CAAAGTGTACATGACAACCTTCGCCGAAGGCAGCGACGCCAGGCTGGAAAAGATC
GTGGAGGGCGACAGCATCAGGAGCGTGAATGAGGGCGAGGCCTTCAGCGCTGAAA
TGGCCGATAAAAACGCCGGCTATAAGATCGGCAACGCCAAATTCAGCCATCCTAA
GGGCTACGCCGTGGTGGCTAACAACCCTCTGTATACAGGACCCGTCCAGCAGGAT
ATGCTCGGCCTGAAGGAAACTCTGGAAAAGAGGTACTTCGGCGAGAGCGCTGATG
GCAATGACAATATTTGTATCCAGGTGATCCATAACATCCTGGACATTGAAAAAAT
CCTCGCCGAATACATTACCAACGCCGCCTACGCCGTCAACAATATCTCCGGCCTG
GATAAGGACATTATTGGATTCGGCAAGTTCTCCACAGTGTATACCTACGACGAAT
TCAAAGACCCCGAGCACCATAGGGCCGCTTTCAACAATAACGATAAGCTCATCAA
CGCCATCAAGGCCCAGTATGACGAGTTCGACAACTTCCTCGATAACCCCAGACTC
GGCTATTTCGGCCAGGCCTTTTTCAGCAAGGAGGGCAGAAATTACATCATCAATT
ACGGCAACGAATGCTATGACATTCTGGCCCTCCTGAGCGGACTGAGGCACTGGGT
GGTCCATAACAACGAAGAAGAGTCCAGGATCTCCAGGACCTGGCTCTACAACCTC
GATAAGAACCTCGACAACGAATACATCTCCACCCTCAACTACCTCTACGACAGGA
TCACCAATGAGCTGACCAACTCCTTCTCCAAGAACTCCGCCGCCAACGTGAACTA
TATTGCCGAAACTCTGGGAATCAACCCTGCCGAATTCGCCGAACAATATTTCAGA
TTCAGCATTATGAAAGAGCAGAAAAACCTCGGATTCAATATCACCAAGCTCAGGG
AAGTGATGCTGGACAGGAAGGATATGTCCGAGATCAGGAAAAATCATAAGGTGTT
CGACTCCATCAGGACCAAGGTCTACACCATGATGGACTTTGTGATTTATAGGTAT
TACATCGAAGAGGATGCCAAGGTGGCTGCCGCCAATAAGTCCCTCCCCGATAATG
AGAAGTCCCTGAGCGAGAAGGATATCTTTGTGATTAACCTGAGGGGCTCCTTCAA
CGACGACCAGAAGGATGCCCTCTACTACGATGAAGCTAATAGAATTTGGAGAAAG
CTCGAAAATATCATGCACAACATCAAGGAATTTAGGGGAAACAAGACAAGAGAGT
ATAAGAAGAAGGACGCCCCTAGACTGCCCAGAATCCTGCCCGCTGGCCGTGATGT
TTCCGCCTTCAGCAAACTCATGTATGCCCTGACCATGTTCCTGGATGGCAAGGAG
ATCAACGACCTCCTGACCACCCTGATTAATAAATTCGATAACATCCAGAGCTTCC
TGAAGGTGATGCCTCTCATCGGAGTCAACGCTAAGTTCGTGGAGGAATACGCCTT
TTTCAAAGACTCCGCCAAGATCGCCGATGAGCTGAGGCTGATCAAGTCCTTCGCT
AGAATGGGAGAACCTATTGCCGATGCCAGGAGGGCCATGTATATCGACGCCATCC
GTATTTTAGGAACCAACCTGTCCTATGATGAGCTCAAGGCCCTCGCCGACACCTT
TTCCCTGGACGAGAACGGAAACAAGCTCAAGAAAGGCAAGCACGGCATGAGAAAT
TTCATTATTAATAACGTGATCAGCAATAAAAGGTTCCACTACCTGATCAGATACG
GTGATCCTGCCCACCTCCATGAGATCGCCAAAAACGAGGCCGTGGTGAAGTTCGT
GCTCGGCAGGATCGCTGACATCCAGAAAAAACAGGGCCAGAACGGCAAGAACCAG
ATCGACAGGTACTACGAAACTTGTATCGGAAAGGATAAGGGCAAGAGCGTGAGCG
AAAAGGTGGACGCTCTCACAAAGATCATCACCGGAATGAACTACGACCAATTCGA
CAAGAAAAGGAGCGTCATTGAGGACACCGGCAGGGAAAACGCCGAGAGGGAGAAG
TTTAAAAAGATCATCAGCCTGTACCTCACCGTGATCTACCACATCCTCAAGAATA
TTGTCAATATCAACGCCAGGTACGTCATCGGATTCCATTGCGTCGAGCGTGATGC
TCAACTGTACAAGGAGAAAGGCTACGACATCAATCTCAAGAAACTGGAAGAGAAG
GGATTCAGCTCCGTCACCAAGCTCTGCGCTGGCATTGATGAAACTGCCCCCGATA
AGAGAAAGGACGTGGAAAAGGAGATGGCTGAAAGAGCCAAGGAGAGCATTGACAG
CCTCGAGAGCGCCAACCCCAAGCTGTATGCCAATTACATCAAATACAGCGACGAG
AAGAAAGCCGAGGAGTTCACCAGGCAGATTAACAGGGAGAAGGCCAAAACCGCCC
TGAACGCCTACCTGAGGAACACCAAGTGGAATGTGATCATCAGGGAGGACCTCCT
GAGAATTGACAACAAGACATGTACCCTGTTCAGAAACAAGGCCGTCCACCTGGAA
GTGGCCAGGTATGTCCACGCCTATATCAACGACATTGCCGAGGTCAATTCCTACT
TCCAACTGTACCATTACATCATGCAGAGAATTATCATGAATGAGAGGTACGAGAA
AAGCAGCGGAAAGGTGTCCGAGTACTTCGACGCTGTGAATGACGAGAAGAAGTAC
AACGATAGGCTCCTGAAACTGCTGTGTGTGCCTTTCGGCTACTGTATCCCCAGGT
TTAAGAACCTGAGCATCGAGGCCCTGTTCGATAGGAACGAGGCCGCCAAGTTCGA
CAAGGAGAAAAAGAAGGTGTCCGGCAATTCCGGATCCggacctaagaaaaagagg
aaggtggcggccgctTACCCATACGATGTTCCAGATTACGCTgctagcTAATCTA
GAgcttatcgataatcaacctctggattacaaaatttgtgaaagattgactggta
ttcttaactatgttgctccttttacgctatgtggatacgctgctttaatgccttt
gtatcatgctattgcttcccgtatggctttcattttctcctccttgtataaatcc
tggttgctgtctctttatgaggagttgtggcccgttgtcaggcaacgtggcgtgg
tgtgcactgtgtttgctgacgcaacccccactggttggggcattgccaccacctg
tcagctcctttccgggactttcgctttccccctccctattgccacggcggaactc
atcgccgcctgccttgcccgctgctggacaggggctcggctgttgggcactgaca
attccgtggtgttgtcggggaaatcatcgtcctttccttggctgctcgcctgtgt
tgccacctggattctgcgcgggacgtccttctgctacgtcccttcggccctcaat
ccagcggaccttccttcccgcggcctgctgccggctctgcggcctcttccgcgtc
ttcgccttcgccctcagacgagtcggatctccctttgggccgcctccccgcatcg
ataccgtcgacctcgagacctagaaaaacatggagcaatcacaagtagcaataca
gcagctaccaatgctgattgtgcctggctagaagcacaagaggaggaggaggtgg
gttttccagtcacacctcaggtacctttaagaccaatgacttacaaggcagctgt
agatcttagccactttttaaaagaaaaggggggactggaagggctaattcactcc
caacgaagacaagatatccttgatctgtggatctaccacacacaaggctacttcc
ctgattggcagaactacacaccagggccagggatcagatatccactgacctttgg
atggtgctacaagctagtaccagttgagcaagagaaggtagaagaagccaatgaa
ggagagaacacccgcttgttacaccctgtgagcctgcatgggatggatgacccgg
agagagaagtattagagtggaggtttgacagccgcctagcatttcatcacatggc
ccgagagctgcatccggactgtactgggtctctctggttagaccagatctgagcc
tgggagctctctggctaactagggaacccactgcttaagcctcaataaagcttgc
cttgagtgcttcaagtagtgtgtgcccgtctgttgtgtgactctggtaactagag
atccctcagacccttttagtcagtgtggaaaatctctagca
>pJW258-pEF-1a-dRfxCas13d-NLS-WPRE;
SEQ ID NO: 6
gggtctctctggttagaccagatctgagcctgggagctctctggctaactaggga
acccactgcttaagcctcaataaagcttgccttgagtgcttcaagtagtgtgtgc
ccgtctgttgtgtgactctggtaactagagatccctcagacccttttagtcagtg
tggaaaatctctagcagtggcgcccgaacagggacttgaaagcgaaagggaaacc
agaggagctctctcgacgcaggactcggcttgctgaagcgcgcacggcaagaggc
gaggggcggcgactggtgagtacgccaaaaattttgactagcggaggctagaagg
agagagatgggtgcgagagcgtcagtattaagcgggggagaattagatcgcgatg
ggaaaaaattcggttaaggccagggggaaagaaaaaatataaattaaaacatata
gtatgggcaagcagggagctagaacgattcgcagttaatcctggcctgttagaaa
catcagaaggctgtagacaaatactgggacagctacaaccatcccttcagacagg
atcagaagaacttagatcattatataatacagtagcaaccctctattgtgtgcat
caaaggatagagataaaagacaccaaggaagctttagacaagatagaggaagagc
aaaacaaaagtaagaccaccgcacagcaagcggccgctgatcttcagacctggag
gaggagatatgagggacaattggagaagtgaattatataaatataaagtagtaaa
aattgaaccattaggagtagcacccaccaaggcaaagagaagagtggtgcagaga
gaaaaaagagcagtgggaataggagctttgttccttgggttcttgggagcagcag
gaagcactatgggcgcagcgtcaatgacgctgacggtacaggccagacaattatt
gtctggtatagtgcagcagcagaacaatttgctgagggctattgaggcgcaacag
catctgttgcaactcacagtctggggcatcaagcagctccaggcaagaatcctgg
ctgtggaaagatacctaaaggatcaacagctcctggggatttggggttgctctgg
aaaactcatttgcaccactgctgtgccttggaatgctagttggagtaataaatct
ctggaacagatttggaatcacacgacctggatggagtgggacagagaaattaaca
attacacaagcttaatacactccttaattgaagaatcgcaaaaccagcaagaaaa
gaatgaacaagaattattggaattagataaatgggcaagtttgtggaattggttt
aacataacaaattggctgtggtatataaaattattcataatgatagtaggaggct
tggtaggtttaagaatagtttttgctgtactttctatagtgaatagagttaggca
gggatattcaccattatcgtttcagacccacctcccaaccccgaggggacccgac
aggcccgaaggaatagaagaagaaggtggagagagagacagagacagatccattc
gattagtgaacggatcggcactgcgtgcgccaattctgcagacaaatggcagtat
tcatccacaattttaaaagaaaaggggggattggggggtacagtgcaggggaaag
aatagtagacataatagcaacagacatacaaactaaagaattacaaaaacaaatt
acaaaaattcaaaattttcgggtttattacagggacagcagagatccagtttggt
taattaatgcaaagatggataaagttttaaacagagaggaatctttgcagctaat
ggaccttctaggtcttgaaaggagtgggaattggctccggtgcccgtcagtgggc
agagcgcacatcgcccacagtccccgagaagttggggggaggggtcggcaattga
accggtgcctagagaaggtggcgcggggtaaactgggaaagtgatgtcgtgtact
ggctccgcctttttcccgagggtgggggagaaccgtatataagtgcagtagtcgc
cgtgaacgttctttttcgcaacgggtttgccgccagaacacaggtaagtgccgtg
tgtggttcccgcgggcctggcctctttacgggttatggcccttgcgtgccttgaa
ttacttccactggctgcagtacgtgattcttgatcccgagcttcgggttggaagt
gggtgggagagttcgaggccttgcgcttaaggagccccttcgcctcgtgcttgag
ttgaggcctggcctgggcgctggggccgccgcgtgcgaatctggtggcaccttcg
cgcctgtctcgctgctttcgataagtctctagccatttaaaatttttgatgacct
gctgcgacgctttttttctggcaagatagtcttgtaaatgcgggccaagatctgc
acactggtatttcggtttttggggccgcgggcggcgacggggcccgtgcgtccca
gcgcacatgttcggcgaggcggggcctgcgagcgcggccaccgagaatcggacgg
gggtagtctcaagctggccggcctgctctggtgcctggcctcgcgccgccgtgta
tcgccccgccctgggcggcaaggctggcccggteggcaecagttgcgtgagcgga
aagatggccgcttcccggccctgctgcagggagctcaaaatggaggacgcggcgc
tcgggagagcgggcgggtgagtcacccacacaaaggaaaagggcctttccgtcct
cagccgtcgcttcatgtgactccacggagtaccgggcgccgtccaggcacctcga
ttagttctcgagcttttggagtacgtcgtctttaggttggggggaggggttttat
gcgatggagtttccccacactgagtgggtggagactgaagttaggccagcttggc
acttgatgtaattctccttggaatttgccctttttgagtttggatcttggttcat
tctcaagcctcagacagtggttcaaagtttttttcttccatttcaggtgtcgtga
cgtacggccaccatgagccccaagaagaagagaaaggtggaggccagcatcgaaa
aaaaaaagtccttcgccaagggcatgggcgtgaagtccacactcgtgtccggctc
caaagtgtacatgacaaccttcgccgaaggcagcgacgccaggctggaaaagatc
gtggagggcgacagcatcaggagcgtgaatgagggcgaggccttcagcgctgaaa
tggccgataaaaacgccggctataagatcggcaacgccaaattcagccatcctaa
gggctacgccgtggtggctaacaaccctctgtatacaggacccgtccagcaggat
atgctcggcctgaaggaaactctggaaaagaggtacttcggcgagagcgctgatg
gcaatgacaatatttgtatccaggtgatccataacatcctggacattgaaaaaat
cctcgccgaatacattaccaacgccgcctacgccgtcaacaatatctccggcctg
gataaggacattattggattcggcaagttctccacagtgtatacctacgacgaat
tcaaagaccccgagcaccatagggccgctttcaacaataacgataagctcatcaa
cgccatcaaggcccagtatgacgagttcgacaacttcctcgataaccccagactc
ggctatttcggccaggcctttttcagcaaggagggcagaaattacatcatcaatt
acggcaacgaatgctatgacattctggccctcctgagcggactggcgcactgggt
ggtcgctaacaacgaagaagagtccaggatctccaggacctggctctacaacctc
gataagaacctcgacaacgaatacatctccaccctcaactacctctacgacagga
tcaccaatgagctgaccaactccttctccaagaactccgccgccaacgtgaacta
tattgccgaaactctgggaatcaaccctgccgaattcgccgaacaatatttcaga
ttcagcattatgaaagagcagaaaaacctcggattcaatatcaccaagctcaggg
aagtgatgctggacaggaaggatatgtccgagatcaggaaaaatcataaggtgtt
cgactccatcaggaccaaggtctacaccatgatggactttgtgatttataggtat
tacatcgaagaggatgccaaggtggctgccgccaataagtccctccccgataatg
agaagtccctgagcgagaaggatatctttgtgattaacctgaggggctccttcaa
cgacgaccagaaggatgccctctactacgatgaagctaatagaatttggagaaag
ctcgaaaatatcatgcacaacatcaaggaatttaggggaaacaagacaagagagt
ataagaagaaggacgcccctagactgcccagaatcctgcccgctggccgtgatgt
ttccgccttcagcaaactcatgtatgccctgaccatgttcctggatggcaaggag
atcaacgacctcctgaccaccctgattaataaattcgataacatccagagcttcc
tgaaggtgatgcctctcatcggagtcaacgctaagttcgtggaggaatacgcctt
tttcaaagactccgccaagatcgccgatgagctgaggctgatcaagtccttcgct
agaatgggagaacctattgccgatgccaggagggccatgtatatcgacgccatcc
gtattttaggaaccaacctgtcctatgatgagctcaaggccctcgccgacacctt
ttccctggacgagaacggaaacaagctcaagaaaggcaagcacggcatgagaaat
ttcattattaataacgtgatcagcaataaaaggttccactacctgatcagatacg
gtgatcctgcccacctccatgagatcgccaaaaacgaggccgtggtgaagttcgt
gctcggcaggatcgctgacatccagaaaaaacagggccagaacggcaagaaccag
atcgacaggtactacgaaacttgtatcggaaaggataagggcaagagcgtgagcg
aaaaggtggacgctctcacaaagatcatcaccggaatgaactacgaccaattcga
caagaaaaggagcgtcattgaggacaccggcagggaaaacgccgagagggagaag
tttaaaaagatcatcagcctgtacctcaccgtgatctaccacatcctcaagaata
ttgtcaatatcaacgccaggtacgtcatcggattccattgcgtcgagcgtgatgc
tcaactgtacaaggagaaaggctacgacatcaatctcaagaaactggaagagaag
ggattcagctccgtcaccaagctctgcgctggcattgatgaaactgcccccgata
agagaaaggacgtggaaaaggagatggctgaaagagccaaggagagcattgacag
cctcgagagcgccaaccccaagctgtatgccaattacatcaaatacagcgacgag
aagaaagccgaggagttcaccaggcagattaacagggagaaggccaaaaccgccc
tgaacgcctacctgaggaacaccaagtggaatgtgatcatcagggaggacctcct
gagaattgacaacaagacatgtaccctgttcgcaaacaaggccgtcgccctggaa
gtggccaggtatgtccacgcctatatcaacgacattgccgaggtcaattcctact
tccaactgtaccattacatcatgcagagaattatcatgaatgagaggtacgagaa
aagcagcggaaaggtgtccgagtacttcgacgctgtgaatgacgagaagaagtac
aacgataggctcctgaaactgctgtgtgtgcctttcggctactgtatccccaggt
ttaagaacctgagcatcgaggccctgttcgataggaacgaggccgccaagttcga
caaggagaaaaagaaggtgtccggcaattccggatccggacctaagaaaaagagg
aaggtggcggccgcttacccatacgatgttccagattacgctgctagcTAATCTA
GAgcttatcgataatcaacctctggattacaaaatttgtgaaagattgactggta
ttcttaactatgttgctccttttacgctatgtggatacgctgctttaatgccttt
gtatcatgctattgcttcccgtatggctttcattttctcctccttgtataaatcc
tggttgctgtctctttatgaggagttgtggcccgttgtcaggcaacgtggcgtgg
tgtgcactgtgtttgctgacgcaacccccactggttggggcattgccaccacctg
tcagctcctttccgggactttcgctttccccctccctattgccacggcggaactc
atcgccgcctgccttgcccgctgctggacaggggctcggctgttgggcactgaca
attccgtggtgttgtcggggaaatcatcgtcctttccttggctgctcgcctgtgt
tgccacctggattctgcgcgggacgtccttctgctacgtcccttcggccctcaat
ccagcggaccttccttcccgcggcctgctgccggctctgcggcctcttccgcgtc
ttcgccttcgccctcagacgagtcggatctccctttgggccgcctccccgcatcg
ataccgtcgacctcgagacctagaaaaacatggagcaatcacaagtagcaataca
gcagctaccaatgctgattgtgcctggctagaagcacaagaggaggaggaggtgg
gttttccagtcacacctcaggtacctttaagaccaatgacttacaaggcagctgt
agatcttagccactttttaaaagaaaaggggggactggaagggctaattcactcc
caacgaagacaagatatccttgatctgtggatctaccacacacaaggctacttcc
ctgattggcagaactacacaccagggccagggatcagatatccactgacctttgg
atggtgctacaagctagtaccagttgagcaagagaaggtagaagaagccaatgaa
ggagagaacacccgcttgttacaccctgtgagcctgcatgggatggatgacccgg
agagagaagtattagagtggaggtttgacagccgcctagcatttcatcacatggc
ccgagagctgcatccggactgtactgggtctctctggttagaccagatctgagcc
tgggagctctctggctaactagggaacccactgcttaagcctcaataaagcttgc
cttgagtgcttcaagtagtgtgtgcccgtctgttgtgtgactctggtaactagag
atccctcagacccttttagtcagtgtggaaaatctctagca
>pJW50-pAAV-pU1a-cMycNLS-dRfxCas13d-GS-cMycNLS-GS-HA-GS-
ADAR2DD(E488Q_T375G)-RBGpA;
SEQ ID NO: 7
cctgcaggcagctgcgcgctcgctcgctcactgaggccgcccgggcaaagcccgg
gcgtcgggcgacctttggtcgcccggcctcagtgagcgagcgagcgcgcagagag
ggagtggccaactccatcactaggggttcctgcggcctctagaatggaggcggta
ctatgtagatgagaattcaggagcaaactgggaaaagcaactgcttccaaatatt
tgtgatttttacagtgtagttttggaaaaactcttagcctaccaattcttctaag
tgttttaaaatgtgggagccagtacacatgaagttatagagtgttttaatgaggc
ttaaatatttaccgtaactatgaaatgctacgcatatcatgctgttcaggctccg
tggccacgcaactcatactaccggtgtcgacgccaccatgcctgcagctaagcgg
gttaagttggatatcgaaaaaaaaaagtccttcgccaagggcatgggcgtgaagt
ccacactcgtgtccggctccaaagtgtacatgacaaccttcgccgaaggcagcga
cgccaggctggaaaagatcgtggagggcgacagcatcaggagcgtgaatgagggc
gaggccttcagcgctgaaatggccgataaaaacgccggctataagatcggcaacg
ccaaattcagccatcctaagggctacgccgtggtggctaacaaccctctgtatac
aggacccgtccagcaggatatgctcggcctgaaggaaactctggaaaagaggtac
ttcggcgagagcgctgatggcaatgacaatatttgtatccaggtgatccataaca
tcctggacattgaaaaaatcctcgccgaatacattaccaacgccgcctacgccgt
caacaatatctccggcctggataaggacattattggattcggcaagttctccaca
gtgtatacctacgacgaattcaaagaccccgagcaccatagggccgctttcaaca
ataacgataagctcatcaacgccatcaaggcccagtatgacgagttcgacaactt
cctcgataaccccagactcggctatttcggccaggcctttttcagcaaggagggc
agaaattacatcatcaattacggcaacgaatgctatgacattctggccctcctga
gcggactggcgcactgggtggtcgctaacaacgaagaagagtccaggatctccag
gacctggctctacaacctcgataagaacctcgacaacgaatacatctccaccctc
aactacctctacgacaggatcaccaatgagctgaccaactccttctccaagaact
ccgccgccaacgtgaactatattgccgaaactctgggaatcaaccctgccgaatt
cgccgaacaatatttcagattcagcattatgaaagagcagaaaaacctcggattc
aatatcaccaagctcagggaagtgatgctggacaggaaggatatgtccgagatca
ggaaaaatcataaggtgttcgactccatcaggaccaaggtctacaccatgatgga
ctttgtgatttataggtattacatcgaagaggatgccaaggtggctgccgccaat
aagtccctccccgataatgagaagtccctgagcgagaaggatatctttgtgatta
acctgaggggctccttcaacgacgaccagaaggatgccctctactacgatgaagc
taatagaatttggagaaagctcgaaaatatcatgcacaacatcaaggaatttagg
ggaaacaagacaagagagtataagaagaaggacgcccctagactgcccagaatcc
tgcccgctggccgtgatgtttccgccttcagcaaactcatgtatgccctgaccat
gttcctggatggcaaggagatcaacgacctcctgaccaccctgattaataaattc
gataacatccagagcttcctgaaggtgatgcctctcatcggagtcaacgctaagt
tcgtggaggaatacgcctttttcaaagactccgccaagatcgccgatgagctgag
gctgatcaagtccttcgctagaatgggagaacctattgccgatgccaggagggcc
atgtatatcgacgccatccgtattttaggaaccaacctgtcctatgatgagctca
aggccctcgccgacaccttttccctggacgagaacggaaacaagctcaagaaagg
caagcacggcatgagaaatttcattattaataacgtgatcagcaataaaaggttc
cactacctgatcagatacggtgatcctgcccacctccatgagatcgccaaaaacg
aggccgtggtgaagttcgtgctcggcaggatcgctgacatccagaaaaaacaggg
ccagaacggcaagaaccagatcgacaggtactacgaaacttgtatcggaaaggat
aagggcaagagcgtgagcgaaaaggtggacgctctcacaaagatcatcaccggaa
tgaactacgaccaattcgacaagaaaaggagcgtcattgaggacaccggcaggga
aaacgccgagagggagaagtttaaaaagatcatcagcctgtacctcaccgtgatc
taccacatcctcaagaatattgtcaatatcaacgccaggtacgtcatcggattcc
attgcgtcgagcgtgatgctcaactgtacaaggagaaaggctacgacatcaatct
caagaaactggaagagaagggattcagctccgtcaccaagctctgcgctggcatt
gatgaaactgcccccgataagagaaaggacgtggaaaaggagatggctgaaagag
ccaaggagagcattgacagcctcgagagcgccaaccccaagctgtatgccaatta
catcaaatacagcgacgagaagaaagccgaggagttcaccaggcagattaacagg
gagaaggccaaaaccgccctgaacgcctacctgaggaacaccaagtggaatgtga
tcatcagggaggacctcctgagaattgacaacaagacatgtaccctgttcgcaaa
caaggccgtcgccctggaagtggccaggtatgtccacgcctatatcaacgacatt
gccgaggtcaattcctacttccaactgtaccattacatcatgcagagaattatca
tgaatgagaggtacgagaaaagcagcggaaaggtgtccgagtacttcgacgctgt
gaatgacgagaagaagtacaacgataggctcctgaaactgctgtgtgtgcctttc
ggctactgtatccccaggtttaagaacctgagcatcgaggccctgttcgatagga
acgaggccgccaagttcgacaaggagaaaaagaaggtgtccggcaattccggatc
ccctgcagctaagcgggttaagttggatggatcctacccatacgatgttccagat
tacgctggatcccagctgcatttaccgcaggttttagctgacgctgtctcacgcc
tggtcctgggtaagtttggtgacctgaccgacaacttctcctcccctcacgctcg
cagaaaagtgctggctggagtcgtcatgacaacaggcacagatgttaaagatgcc
aaggtgataagtgtttctacaggaggcaaatgtattaatggtgaatacatgagtg
atcgtggccttgcattaaatgactgccatgcagaaataatatctcggagatcctt
gctcagatttctttatacacaacttgagctttacttaaataacaaagatgatcaa
aaaagatccatctttcagaaatcagagcgaggggggtttaggctgaaggagaatg
tccagtttcatctgtacatcagcacctctccctgtggagatgccagaatcttctc
accacatgagccaatcctggaagaaccagcagatagacacccaaatcgtaaagca
agaggacagctacggaccaaaatagagtctggtcaggggacgattccagtgcgct
ccaatgcgagcatccaaacgtgggacggggtgctgcaaggggagcggctgctcac
catgtcctgcagtgacaagattgcacgctggaacgtggtgggcatccagggatca
ctgctcagcattttcgtggagcccatttacttctcgagcatcatcctgggcagcc
tttaccacggggaccacctttccagggccatgtaccagcggatctccaacataga
ggacctgccacctctctacaccctcaacaagcctttgctcagtggcatcagcaat
gcagaagcacggcagccagggaaggcccccaacttcagtgtcaactggacggtag
gcgactccgctattgaggtcatcaacgccacgactgggaaggatgagctgggccg
cgcgtcccgcctgtgtaagcacgcgttgtactgtcgctggatgcgtgtgcacggc
aaggttccctcccacttactacgctccaagattaccaagcccaacgtgtaccatg
agtccaagctggcggcaaaggagtaccaggccgccaaggcgcgtctgttcacagc
cttcatcaaggcggggctgggggcctgggtggagaagcccaccgagcaggaccag
ttctcactcacgtaaaagcttgaattcgatctttttccctctgccaaaaattatg
gggacatcatgaagccccttgagcatctgacttctggctaataaaggaaatttat
tttcattgcaatagtgtgttggaattttttgtgtctctcactcggcggccgcagg
aacccctagtgatggagttggccactccctctctgcgcgctcgctcgctcactga
ggccgggcgaccaaaggtcgcccgacgcccgggctttgcccgggcggcctcagtg
agcgagcgagcgcgcagctgcctgcagg
>Rfx-Cas13d;
SEQ ID NO: 8
MSPKKKRKVEASIEKKKSFAKGMGVKSTLVSGSKVYMTTFAEGSDARLEKIVEGD
SIRSVNEGEAFSAEMADKNAGYKIGNAKFSHPKGYAVVANNPLYTGPVQQDMLGL
KETLEKRYFGESADGNDNICIQVIHNILDIEKILAEYITNAAYAVNNISGLDKDI
IGFGKFSTVYTYDEFKDPEHHRAAFNNNDKLINAIKAQYDEFDNFLDNPRLGYFG
QAFFSKEGRNYIINYGNECYDILALLSGLRHWVVHNNEEESRISRTWLYNLDKNL
DNEYISTLNYLYDRITNELTNSFSKNSAANVNYIAETLGINPAEFAEQYFRFSIM
KEQKNLGFNITKLREVMLDRKDMSEIRKNHKVFDSIRTKVYTMMDFVIYRYYIEE
DAKVAAANKSLPDNEKSLSEKDIFVINLRGSFNDDQKDALYYDEANRIWRKLENI
MHNIKEFRGNKTREYKKKDAPRLPRILPAGRDVSAFSKLMYALTMFLDGKEINDL
LTTLINKFDNIQSFLKVMPLIGVNAKFVEEYAFFKDSAKIADELRLIKSFARMGE
PIADARRAMYIDAIRILGTNLSYDELKALADTFSLDENGNKLKKGKHGMRNFIIN
NVISNKRFHYLIRYGDPAHLHEIAKNEAVVKFVLGRIADIQKKQGQNGKNQIDRY
YETCIGKDKGKSVSEKVDALTKIITGMNYDQFDKKRSVIEDTGRENAEREKFKKI
ISLYLTVIYHILKNIVNINARYVIGFHCVERDAQLYKEKGYDINLKKLEEKGFSS
VTKLCAGIDETAPDKRKDVEKEMAERAKESIDSLESANPKLYANYIKYSDEKKAE
EFTRQINREKAKTALNAYLRNTKWNVIIREDLLRIDNKTCTLFRNKAVHLEVARY
VHAYINDIAEVNSYFQLYHYIMQRIIMNERYEKSSGKVSEYFDAVNDEKKYNDRL
LKLLCVPFGYCIPRFKNLSIEALFDRNEAAKFDKEKKKVSGNS
>Rfx-dCas13d;
SEQ ID NO: 9
MSPKKKRKVEASIEKKKSFAKGMGVKSTLVSGSKVYMTTFAEGSDARLEKIVEGD
SIRSVNEGEAFSAEMADKNAGYKIGNAKFSHPKGYAVVANNPLYTGPVQQDMLGL
KETLEKRYFGESADGNDNICIQVIHNILDIEKILAEYITNAAYAVNNISGLDKDI
IGFGKFSTVYTYDEFKDPEHHRAAFNNNDKLINAIKAQYDEFDNFLDNPRLGYFG
QAFFSKEGRNYIINYGNECYDILALLSGLAHWVVANNEEESRISRTWLYNLDKNL
DNEYISTLNYLYDRITNELTNSFSKNSAANVNYIAETLGINPAEFAEQYFRFSIM
KEQKNLGFNITKLREVMLDRKDMSEIRKNHKVFDSIRTKVYTMMDFVIYRYYIEE
DAKVAAANKSLPDNEKSLSEKDIFVINLRGSFNDDQKDALYYDEANRIWRKLENI
MHNIKEFRGNKTREYKKKDAPRLPRILPAGRDVSAFSKLMYALTMFLDGKEINDL
LTTLINKFDNIQSFLKVMPLIGVNAKFVEEYAFFKDSAKIADELRLIKSFARMGE
PIADARRAMYIDAIRILGTNLSYDELKALADTFSLDENGNKLKKGKHGMRNFIIN
NVISNKRFHYLIRYGDPAHLHEIAKNEAVVKFVLGRIADIQKKQGQNGKNQIDRY
YETCIGKDKGKSVSEKVDALTKIITGMNYDQFDKKRSVIEDTGRENAEREKFKKI
ISLYLTVIYHILKNIVNINARYVIGFHCVERDAQLYKEKGYDINLKKLEEKGFSS
VTKLCAGIDETAPDKRKDVEKEMAERAKESIDSLESANPKLYANYIKYSDEKKAE
EFTRQINREKAKTALNAYLRNTKWNVIIREDLLRIDNKTCTLFANKAVALEVARY
VHAYINDIAEVNSYFQLYHYIMQRIIMNERYEKSSGKVSEYFDAVNDEKKYNDRL
LKLLCVPFGYCIPRFKNLSIEALFDRNEAAKFDKEKKKVSGNS

### Other Embodiments

Embodiment 1. An isolated nucleic acid comprising an expression cassette encoding a pre-trans-splicing (PTS) molecule, wherein the PTS molecule comprises: i) one or more guideRNAs (gRNAs) that target an intron-exon boundary; ii) an intronic sequence having a splice signal; and iii) a donor sequence encoding a gene product of a gene of interest, or portion thereof.

Embodiment 2. The isolated nucleic acid of embodiment 1, wherein the gRNAs comprise one or more Cas13 direct repeats.

Embodiment 3. The isolated nucleic acid of embodiment 1 or 2, wherein the splice signal is a branch point sequence (BPS).

Embodiment 4. The isolated nucleic acid of any one of embodiments 1 to 3, wherein the donor sequence of (iii) is an exonic sequence, optionally wherein the donor sequence of (iii) comprises an entire exon of the gene of interest.

Embodiment 5. The isolated nucleic acid of any one of embodiments 1 to 4, wherein the expression cassette comprises a promoter operably linked to the nucleic acid sequence encoding the PTS molecule.

Embodiment 6. The isolated nucleic acid of embodiment 5, wherein the promoter is an RNA polymerase II (pol II) promoter, optionally wherein the promoter is a chicken beta-actin (CB) promoter.

Embodiment 7. The isolated nucleic acid of embodiment 5 or 6, wherein the promoter is an inducible promoter or a tissue-specific promoter.

Embodiment 8. The isolated nucleic acid of any one of embodiments 1 to 7, wherein the one or more gRNAs of (i) and the intronic sequence of (ii) are adjacent to one another.

Embodiment 9. The isolated nucleic acid of embodiment 8, wherein the one or more gRNAs are positioned 5′ relative to the intronic sequence.

Embodiment 10. The isolated nucleic acid of embodiment 8, wherein the one or more gRNAs are positioned 3′ relative to the intronic sequence.

Embodiment 11. The isolated nucleic acid of embodiment 9, wherein the donor sequence of (iii) is positioned 3′ relative to the intronic sequence.

Embodiment 12. The isolated nucleic acid of embodiment 10, wherein the donor sequence of (iii) is positioned 5′ relative to the intronic sequence.

Embodiment 13. The isolated nucleic acid of any one of embodiments 1 to 12, wherein the expression construct further comprises: (iv) a sequence encoding a self-cleaving ribozyme, optionally a hammerhead ribozyme.

Embodiment 14. The isolated nucleic acid of embodiment 13, wherein the ribozyme-encoding sequence is positioned 5′ to the one or more gRNAs.

Embodiment 15. The isolated nucleic acid of any one of embodiments 1 to 14, wherein the expression construct further comprises: (v) a sequence encoding an RNA-guided nuclease; optionally, wherein the RNA-guided nuclease is a Cas13 nuclease.

Embodiment 16. The isolated nucleic acid of embodiment 15, wherein the Cas13 nuclease is selected from the group consisting of Cas13b, Cas13d, and dCas13d nuclease.

Embodiment 17. The isolated nucleic acid of embodiment 15 or 16, wherein the sequence encoding the RNA-guided nuclease is operably linked to a nuclear localization signal (NLS) sequence.

Embodiment 18. The isolated nucleic acid of any one of embodiments 1 to 17, wherein the expression construct further comprises: (vi) a sequence encoding an adenosine deaminase domain, optionally wherein the deaminase domain is an ADAR deaminase domain (ADARDD).

Embodiment 19. The isolated nucleic acid of any one of embodiments 1 to 18, wherein the expression cassette is flanked by viral vector repeat sequences.

Embodiment 20. The isolated nucleic acid of embodiment 19, wherein the viral vector repeat sequences are adeno-associated virus (AAV) inverted terminal repeats (ITRs).

Embodiment 21. A composition comprising the isolated nucleic acid of any one of embodiments 1 to 20.

Embodiment 22. The composition of embodiment 21, wherein the composition further comprises an isolated nucleic acid encoding an RNA-guided nuclease, optionally wherein the RNA-guided nuclease is a Cas13 nuclease.

Embodiment 23. The composition of embodiment 22, wherein the Cas13 nuclease is selected from the group consisting of Cas13b, Cas13d, and dCas13d nuclease.

Embodiment 24. The composition of embodiment 22 or 23, wherein the nucleic acid sequence encoding the RNA-guided nuclease is operably linked to a nuclear localization signal (NLS) sequence.

Embodiment 25. The composition of any one of embodiments 21 to 24, wherein the composition further comprises an isolated nucleic acid encoding an adenosine deaminase domain, optionally wherein the deaminase domain is an ADAR deaminase domain (ADARDD).

Embodiment 26. A composition comprising: (i) a first recombinant adeno-associated virus (rAAV) particle comprising the isolated nucleic acid of any one of embodiments 1 to 14; and (ii) a second rAAV particle encoding an RNA-guided nuclease, optionally wherein the RNA-guided nuclease is a Cas13 nuclease.

Embodiment 27. The composition of embodiment 26, wherein the Cas13 nuclease is selected from the group consisting of Cas13b, Cas13d, and dCas13d nuclease.

Embodiment 28. The composition of embodiment 26 or 27, wherein the second rAAV encodes an adenosine deaminase domain, optionally wherein the deaminase domain is an ADAR deaminase domain (ADARDD).

Embodiment 29. A method for replacing a mutant exon of a gene of interest in a cell, the method comprising expressing in a cell having a mutant exon of a gene of interest: (i) the isolated nucleic acid of any one of embodiments 1 to 14, wherein one or more of the gRNAs encoded by the isolated nucleic acid specifically bind to an intron-exon boundary that is 5′ relative to the mutant exon of the gene of interest; and (ii) an RNA-guided nuclease; wherein the donor sequence of the isolated nucleic acid encodes a wild-type exon of the gene of interest corresponding to the mutant exon.

Embodiment 30. The method of embodiment 29, wherein the mutant exon comprises one or more nucleic acid substitutions, insertions, or deletions relative to the wild-type exon.

Embodiment 31. The method of embodiment 29 or 30, wherein the cell is a mammalian cell, optionally wherein the mammalian cell is a human cell.

Embodiment 32. The method of any one of embodiments 29 to 31, wherein the cell is in a subject, optionally wherein the subject is a human.

Embodiment 33. The method of any one of embodiments 29 to 32, wherein the gene of interest is DMD, CFTR, SMN1, SMN2, MECP2, or IDUA.

Embodiment 34. The method of any one of embodiments 29 to 33, wherein the RNA-guided nuclease is a Cas13 nuclease, optionally Cas13b or Cas13d nuclease.

Embodiment 35. The method of embodiment 34, wherein the Cas13 nuclease is a dead Cas13 (dCas13) nuclease, optionally dCas13d nuclease.

Embodiment 36. The method of embodiment 34 or 35, wherein the Cas13 nuclease is fused to a ADARDD domain.

Embodiment 37. The method of any one of embodiments 29 to 26, wherein the isolated nucleic acid of (i) and/or the RNA-guided nuclease of (ii) is expressed by an rAAV.

Embodiment 38. The method of any one of embodiments 29 to 26, wherein expression of the isolated nucleic acid of (i) and the RNA-guided nuclease of (ii) results in translation of a full-length, wild-type gene product of the gene of interest.

Embodiment 39. A method for treating a disease associated with a loss of protein function in a subject in need thereof, the method comprising administering to the subject: (i) the isolated nucleic acid of any one of embodiments 1 to 14, wherein one or more of the gRNAs encoded by the isolated nucleic acid specifically bind to an intron-exon boundary that is 5′ relative to a mutant exon of a gene of interest; and (ii) an RNA-guided nuclease; wherein the subject has a disease characterized by the presence of the mutant exon in the gene of interest, and wherein the donor sequence of the isolated nucleic acid encodes a wild-type exon of the gene of interest corresponding to the mutant exon.

Embodiment 40. A method for treating a disease associated with a dominant negative protein function in a subject in need thereof, the method comprising administering to the subject: (i) the isolated nucleic acid of any one of claims 1 to 14, wherein one or more of the gRNAs encoded by the isolated nucleic acid specifically bind to an intron-exon boundary that is 5′ relative to a mutant exon of a gene of interest; and (ii) an RNA-guided nuclease; wherein the subject has a disease characterized by the presence of the mutant exon in the gene of interest, and wherein the donor sequence of the isolated nucleic acid encodes a wild-type exon of the gene of interest corresponding to the mutant exon.

Embodiment 41. A method for treating a disease associated with a gain of function protein function in a subject in need thereof, the method comprising administering to the subject: (i) the isolated nucleic acid of any one of claims 1 to 14, wherein one or more of the gRNAs encoded by the isolated nucleic acid specifically bind to an intron-exon boundary that is 5′ relative to a mutant exon of a gene of interest; and (ii) an RNA-guided nuclease; wherein the subject has a disease characterized by the presence of the mutant exon in the gene of interest, and wherein the donor sequence of the isolated nucleic acid encodes a wild-type exon of the gene of interest corresponding to the mutant exon.

Embodiment 42. The method of any one of claims 39-41, wherein the mutant exon is located in one of the following genes: DMD, CFTR, SMN1, SMN2, MECP2, or IDUA.

Embodiment 43. The method of claim 42, wherein the mutant exon comprises one or more nucleic acid substitutions, insertions, or deletions relative to the wild-type exon.

Embodiment 44. The method of any one of claims 39-43, wherein the subject is a human.

Embodiment 45. The method of any one of claims 39-44, wherein the RNA-guided nuclease is a Cas13 nuclease, optionally wherein the Cas13 nuclease is Cas13b, Cas13d, or dCas13d.

Embodiment 46. The method of any one of claims 39-45, wherein the isolated nucleic acid of (i) and/or the RNA-guided nuclease of (ii) is administered to the subject via an rAAV.

Embodiment 47. The method of any one of claims 39-46, wherein administration of the isolated nucleic acid of (i) and the RNA-guided nuclease of (ii) results in translation of a full-length, wild-type gene product of the gene of interest.

Embodiment 48. An isolated nucleic acid comprising a pre-trans-splicing (PTS) molecule, wherein the PTS molecule comprises: i) one or more guideRNAs (gRNAs) that target an intron-exon boundary; ii) an intronic sequence having a splice signal; and iii) a donor sequence encoding a gene product of a gene of interest, or portion thereof.

In addition to the embodiments expressly described herein, it is to be understood that all of the features disclosed in this disclosure may be combined in any combination (e.g., permutation, combination). Each element disclosed in the disclosure may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, and can make various changes and modifications of the invention to adapt it to various usages and conditions. Thus, other embodiments are also within the claims.

What is claimed is:

1. An isolated nucleic acid comprising an expression cassette encoding a pre-trans-splicing (PTS) molecule, wherein the PTS molecule comprises:
i) one or more guideRNAs (gRNAs) that target an intron-exon boundary;
ii) an intronic sequence having a splice signal; and
iii) a donor sequence encoding a gene product of a gene of interest, or portion thereof.

2. The isolated nucleic acid of claim 1, wherein each of the one or more gRNAs comprise one or more Cas13 direct repeats.

3. The isolated nucleic acid of claim 1 or 2, wherein the splice signal comprises a branch point sequence (BPS).

4. The isolated nucleic acid of any one of claims 1 to 3, wherein the donor sequence of (iii) is an exonic sequence, optionally wherein the donor sequence of (iii) comprises an entire exon of the gene of interest, optionally, wherein the donor sequence of (iii) comprises more than one entire exons of a gene of interest.

5. The isolated nucleic acid of any one of claims 1 to 4, wherein the expression cassette comprises a promoter operably linked to the nucleic acid sequence encoding the PTS molecule.

6. The isolated nucleic acid of claim 5, wherein the promoter is an RNA polymerase II (pol II) promoter, optionally wherein the promoter is a chicken beta-actin (CB) promoter.

7. The isolated nucleic acid of claim 5 or 6, wherein the promoter is an inducible promoter or a tissue-specific promoter.

8. The isolated nucleic acid of any one of claims 1 to 7, wherein the one or more gRNAs of (i) and the intronic sequence of (ii) are adjacent to one another.

9. The isolated nucleic acid of claim 8, wherein the one or more gRNAs are positioned 5′ relative to the intronic sequence.

10. The isolated nucleic acid of claim 8, wherein the one or more gRNAs are positioned 3′ relative to the intronic sequence.

11. The isolated nucleic acid of claim 9, wherein the donor sequence of (iii) is positioned 3′ relative to the intronic sequence.

12. The isolated nucleic acid of claim 10, wherein the donor sequence of (iii) is positioned 5′ relative to the intronic sequence.

13. The isolated nucleic acid of any one of claims 1 to 12, wherein the expression construct further comprises:
(iv) a sequence encoding a self-cleaving ribozyme, optionally a hammerhead ribozyme.

14. The isolated nucleic acid of claim 13, wherein the ribozyme-encoding sequence is positioned 5′ to the one or more gRNAs.

15. The isolated nucleic acid of any one of claims 1 to 14, wherein the expression construct further comprises:
(v) a sequence encoding an RNA-guided nuclease;
optionally, wherein the RNA-guided nuclease is a Cas13 nuclease.

16. The isolated nucleic acid of claim 15, wherein the Cas13 nuclease is selected from the group consisting of Cas13b, Cas13d, and dCas13d nuclease.

17. The isolated nucleic acid of claim 15 or 16, wherein the sequence encoding the RNA-guided nuclease is operably linked to a nuclear localization signal (NLS) sequence.

18. The isolated nucleic acid of any one of claims 1 to 17, wherein the expression construct further comprises:
(vi) a sequence encoding an adenosine deaminase domain, optionally, wherein the deaminase domain is an ADAR deaminase domain (ADARDD).

19. The isolated nucleic acid of any one of claims 1 to 18, wherein the expression cassette is flanked by viral vector repeat sequences.

20. The isolated nucleic acid of claim 19, wherein the viral vector repeat sequences are adeno-associated virus (AAV) inverted terminal repeats (ITRs).

21. A composition comprising the isolated nucleic acid of any one of claims 1 to 20.

22. The composition of claim 21, wherein the composition further comprises an isolated nucleic acid encoding an RNA-guided nuclease, optionally wherein the RNA-guided nuclease is a Cas13 nuclease.

23. The composition of claim 22, wherein the Cas13 nuclease is selected from the group consisting of Cas13b, Cas13d, and dCas13d nuclease.

24. The composition of claim 22 or 23, wherein the nucleic acid sequence encoding the RNA-guided nuclease is operably linked to a nuclear localization signal (NLS) sequence.

25. The composition of any one of claims 21 to 24, wherein the composition further comprises an isolated nucleic acid encoding an adenosine deaminase domain, optionally wherein the deaminase domain is an ADAR deaminase domain (ADARDD).

26. A composition comprising:
(i) a first recombinant adeno-associated virus (rAAV) particle comprising the isolated nucleic acid of any one of claims 1 to 14; and
(ii) a second rAAV particle encoding an RNA-guided nuclease, optionally, wherein the RNA-guided nuclease is a Cas13 nuclease.

27. The composition of claim 26, wherein the Cas13 nuclease is selected from the group consisting of Cas13b, Cas13d, and dCas13d nuclease.

28. The composition of claim 26 or 27, wherein the second rAAV encodes an adenosine deaminase domain, optionally wherein the deaminase domain is an ADAR deaminase domain (ADARDD).

29. A method for replacing a mutant exon of a gene of interest in a cell, the method comprising expressing in a cell having a mutant exon of a gene of interest:
(i) the isolated nucleic acid of any one of claims 1 to 14, wherein one or more of the gRNAs encoded by the isolated nucleic acid specifically bind to an intron-exon boundary that is 5′ relative to the mutant exon of the gene of interest; and
(ii) an RNA-guided nuclease;
wherein the donor sequence of the isolated nucleic acid encodes a wild-type exon of the gene of interest corresponding to the mutant exon.

30. The method of claim 29, wherein the mutant exon comprises one or more nucleic acid substitutions, insertions, or deletions relative to the wild-type exon.

31. The method of claim 29 or 30, wherein the cell is a mammalian cell, optionally wherein the mammalian cell is a human cell.

32. The method of any one of claims 29 to 31, wherein the cell is in a subject, optionally wherein the subject is a human.

33. The method of any one of claims 29 to 32, wherein the gene of interest is DMD, CFTR, SMN1, SMN2, MECP2, or IDUA.

34. The method of any one of claims 29 to 33, wherein the RNA-guided nuclease is a Cas13 nuclease, optionally Cas13b or Cas13d nuclease.

35. The method of claim 34, wherein the Cas13 nuclease is a dead Cas13 (dCas13) nuclease, optionally dCas13d nuclease.

36. The method of claim 34 or 35, wherein the Cas13 nuclease is fused to a ADARDD domain.

37. The method of any one of claims 29 to 26, wherein the isolated nucleic acid of (i) and/or the RNA-guided nuclease of (ii) is expressed by an rAAV.

38. The method of any one of claims 29 to 26, wherein expression of the isolated nucleic acid of (i) and the RNA-guided nuclease of (ii) results in translation of a full-length, wild-type gene product of the gene of interest.

39. A method for treating a disease associated with a loss of protein function in a subject in need thereof, the method comprising administering to the subject:
(i) the isolated nucleic acid of any one of claims 1 to 14, wherein one or more of the gRNAs encoded by the isolated nucleic acid specifically bind to an intron-exon boundary that is 5′ relative to a mutant exon of a gene of interest; and
(ii) an RNA-guided nuclease;
wherein the subject has a disease characterized by the presence of the mutant exon in the gene of interest, and wherein the donor sequence of the isolated nucleic acid encodes a wild-type exon of the gene of interest corresponding to the mutant exon.

40. A method for treating a disease associated with a dominant negative protein function in a subject in need thereof, the method comprising administering to the subject:
(i) the isolated nucleic acid of any one of claims 1 to 14, wherein one or more of the gRNAs encoded by the isolated nucleic acid specifically bind to an intron-exon boundary that is 5′ relative to a mutant exon of a gene of interest; and
(ii) an RNA-guided nuclease;
wherein the subject has a disease characterized by the presence of the mutant exon in the gene of interest, and wherein the donor sequence of the isolated nucleic acid encodes a wild-type exon of the gene of interest corresponding to the mutant exon.

41. A method for treating a disease associated with a gain of function protein function in a subject in need thereof, the method comprising administering to the subject:
(i) the isolated nucleic acid of any one of claims 1 to 14, wherein one or more of the gRNAs encoded by the isolated nucleic acid specifically bind to an intron-exon boundary that is 5′ relative to a mutant exon of a gene of interest; and
(ii) an RNA-guided nuclease;
wherein the subject has a disease characterized by the presence of the mutant exon in the gene of interest, and wherein the donor sequence of the isolated nucleic acid encodes a wild-type exon of the gene of interest corresponding to the mutant exon.

42. The method of any one of claims 39-41, wherein the mutant exon is located in one of the following genes: DMD, CFTR, SMN1, SMN2, MECP2, or IDUA.

43. The method of claim 42, wherein the mutant exon comprises one or more nucleic acid substitutions, insertions, or deletions relative to the wild-type exon.

44. The method of any one of claims 39-43, wherein the subject is a human.

45. The method of any one of claims 39-44, wherein the RNA-guided nuclease is a Cas13 nuclease, optionally wherein the Cas13 nuclease is Cas13b, Cas13d, or dCas13d.

46. The method of any one of claims 39-45, wherein the isolated nucleic acid of (i) and/or the RNA-guided nuclease of (ii) is administered to the subject via an rAAV.

47. The method of any one of claims 39-46, wherein administration of the isolated nucleic acid of (i) and the RNA-guided nuclease of (ii) results in translation of a full-length, wild-type gene product of the gene of interest.

48. An isolated nucleic acid comprising a pre-trans-splicing (PTS) molecule, wherein the PTS molecule comprises:
i) one or more guideRNAs (gRNAs) that target an intron-exon boundary;
ii) an intronic sequence having a splice signal; and
iii) a donor sequence encoding a gene product of a gene of interest, or portion thereof.
